(19) United States
(12) Patent Application Publication
(10) Pub. No.: US 2023/0148849 A1
(43) Pub. Date: May 18, 2023

(54) MEDICAL SYSTEM AND CANNULATION METHOD

(71) Applicant: OLYMPUS CORPORATION, Tokyo (JP)

(72) Inventors: Shintaro INOUE, Cambridge, MA (US); Kazuhiro GONO, Sagamihara-shi (JP); Nobuko OKUMURA, Tokyo (JP); Takashi OTAWARA, Tokyo (JP); Kazuki MINAMIMOTO, Ebina-shi (JP); Anthony PIROZZI, Raleigh, NC (US)

(73) Assignee: OLYMPUS CORPORATION, Tokyo (JP)

(21) Appl. No.: 17/989,146

(22) Filed: Nov. 17, 2022

Related U.S. Application Data
(60) Provisional application No. 63/280,716, filed on Nov. 18, 2021.
(60) Provisional application No. 63/294,453, filed on Dec. 29, 2021.
(60) Provisional application No. 63/294,482, filed on Dec. 29, 2021.

(51) Int. Cl. A61B 1/018; A61B 1/00

(52) U.S. Cl. CPC A61B 1/018; A61B 1/00082

(57) ABSTRACT

A medical system including an endoscope configured to electrically drive an endoscopic operation. The endoscopic operation includes at least one of a forward and backward movement of an insertion section, a bending angle of a bending section of the insertion section, and a rolling rotation of the insertion section, the endoscope being configured to capture an endoscope image. The medical system further includes a processor comprising hardware. The processor is configured to control the endoscopic operation to achieve a second positioning subsequent to a first positioning, the first positioning being where the insertion section is positioned with respect to a papillary portion of a duodenum, the second positioning positions a distal end section of the insertion section with respect to a papillary portion of the duodenum based on the endoscope image.

### CROSS-REFERENCE TO RELATED APPLICATIONS

This application claims the benefit of priority to U.S. Provisional Application Nos. 63/280,716 filed Nov. 18, 2021; 63/294,453 filed Dec. 29, 2021; and 63/294,482 filed Dec. 29, 2021, the entire contents of each of which is incorporated herein by reference.

### BACKGROUND

A technique called endoscopic retrograde cholangiopancreatography (ERCP) is known that captures an X-ray image or a CT image of biliary duct by inserting a cannula into a biliary duct from a treatment tool channel of an endoscope, injecting a contrast agent from the cannula, and performing X-ray imaging or CT imaging. A robotic catheter system for performing ERCP by remotely operating a catheter system is also known.

In the prior art, an electric catheter is known to be operated by an operator workstation or controller by an operator in a robotic catheter system using a catheter whose operation is electrically driven. The operator workstation is so-called a console-type operation device, equipped with buttons, levers, etc. for example. The operator is seated in front of the console and operates the buttons, levers, etc. The controller is a handy operation device equipped with, for example, dials, D-pad, buttons, etc. The operator holds the controller and operates the dials, the D-pad, buttons, etc.

### SUMMARY

Accordingly, there is provided a medical system comprising: an endoscope configured to electrically drive an endoscopic operation, the endoscopic operation comprising at least one of a forward and backward movement of an insertion section, a bending angle of a bending section of the insertion section, and a rolling rotation of the insertion section, the endoscope being configured to capture an endoscope image and a processor comprising hardware, the processor being configured to control the endoscopic operation to achieve a second positioning subsequent to a first positioning, the first positioning being where the insertion section is positioned with respect to a papillary portion of a duodenum, the second positioning positions a distal end section of the insertion section with respect to a papillary portion of the duodenum based on the endoscope image.

### BRIEF DESCRIPTION OF THE DRAWINGS

FIG. 1 shows organs and tissues involved in the ERCP procedure.

FIG. 2 shows a flow of the ERCP procedure.

FIG. 3 shows a schematic diagram of the form of papillary portion viewed directly from the front, and examples of individual differences in shape.

FIG. 4 is a cross-sectional view showing the form of a luminal tissue and its opening.

FIG. 5 shows a basic configuration example of a medical system according to the present embodiment.

FIG. 6 shows a first flow of the procedure according to the present embodiment.

FIGS. 7a and 7b show a comparison between a case without an overtube and a case with an overtube.

FIG. 8 shows the vicinity of the distal end of an endoscope positioned by an overtube and a balloon.

FIG. 9 shows a detailed configuration example of a medical system.

FIG. 10 shows a detailed configuration example of a drive control device.

FIG. 11 is a schematic view of an endoscope including a bending section and a driving mechanism thereof.

FIGS. 12a-12c show a detailed configuration example of a forward/backward drive device.

FIG. 13 is a perspective view of a connecting section including a rolling drive device.

FIGS. 14a and 14b show a detailed configuration example of a distal end section of an endoscope including a raising base of a treatment tool.

FIG. 15 shows a detailed configuration example of a treatment tool.

FIGS. 16a and 16b show a configuration example of a drive system of an overtube.

FIG. 17 shows a configuration example of a drive system of a balloon.

FIG. 18 shows a first modification of a holding member.

FIG. 19 shows a second modification of a holding member.

FIG. 20 shows an organ structure of a patient who had a gastric bypass surgery according to the Roux-en Y method.

FIG. 21 shows a modification of an operation device for manually operating an electric endoscope.

FIG. 22 is a diagram showing an operation of inserting a cannula when using non-electric endoscope and cannula.

FIG. 23 shows a basic configuration example of a medical system according to another embodiment.

FIG. 24 shows a detailed configuration example of an electric treatment tool.

FIG. 25 shows a first detailed configuration example of a treatment tool operation device.

FIG. 26 shows a block configuration example of a medical system.

FIG. 27 shows a first detailed configuration example of a first base.

FIG. 28 shows a second detailed configuration example of the first base.

FIG. 29 shows an example of control of a force feedback.

FIG. 30 shows an example of control of force feedback using an image.

FIG. 31 shows a flow of ERCP procedure using a medical system of the other embodiment.

FIG. 32 shows a flow in the case of enabling/disabling functions based on the type of treatment tool.

FIG. 33 shows a second detailed configuration example of the treatment tool operation device.

FIGS. 34a and 34b show a third detailed configuration example of the treatment tool operation device.

FIG. 35 shows a detailed configuration example of a medical system.

FIG. 36 shows the vicinity of the distal end of an endoscope positioned at a papillary portion of duodenum.

FIGS. 37a-37c show a detailed configuration example of a forward/backward drive device.

FIGS. 38a and 38b show a detailed configuration example of a distal end section of an endoscope including a raising base of a treatment tool.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

Explanation of ERCP

The present embodiment relates to automatic control when performing ERCP using an electric medical system. ERCP stands for Endoscopic Retrograde Cholangiopancreatography. First, before describing the present embodiment, the details of procedure of ERCP is described below.

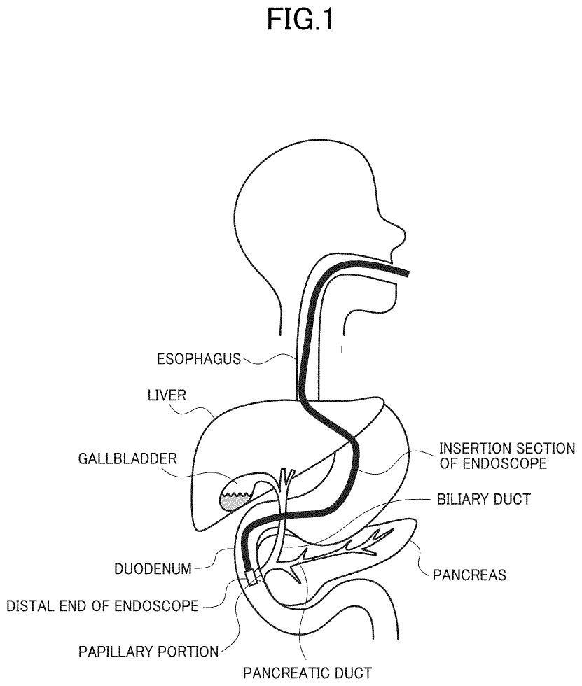

FIG. 1 shows organs and tissues involved in the ERCP procedure. The organs include multiple types of tissues, forming a unique structure with a specific function. In FIG. 1, the liver, gallbladder, pancreas, esophagus, stomach, and duodenum are shown as organs. Tissues are formed by related cells combined, and examples include blood vessels, muscles, skin, and the like. In FIG. 1, a biliary duct and a pancreatic duct are shown as tissues.

The biliary duct is the target of the ERCP procedure. The biliary duct is a pipeline for allowing the bile produced in the liver to flow into the duodenum. When approaching the biliary duct using an endoscope, a treatment tool inserted into the channel of the endoscope is inserted to the biliary duct from the papillary portion of the duodenum while holding the endoscope at the position of the duodenum. Hereinafter, the papillary portion of the duodenum is simply referred to as a papillary portion. The papillary portion is a region including an opening of the luminal tissue with respect to the duodenum. Not only the opening but also the structure around the opening is referred to as a papillary portion. The opening of the luminal tissue is the opening of a common duct with respect to the duodenum. The common duct is formed as the confluence of the biliary duct and pancreatic duct. However, as described later, the papillary portion largely varies between individuals. For example, in some cases, the biliary duct opens directly to the duodenum without being merged with the pancreatic duct. In this case, the opening of the luminal tissue is the opening of the biliary duct.

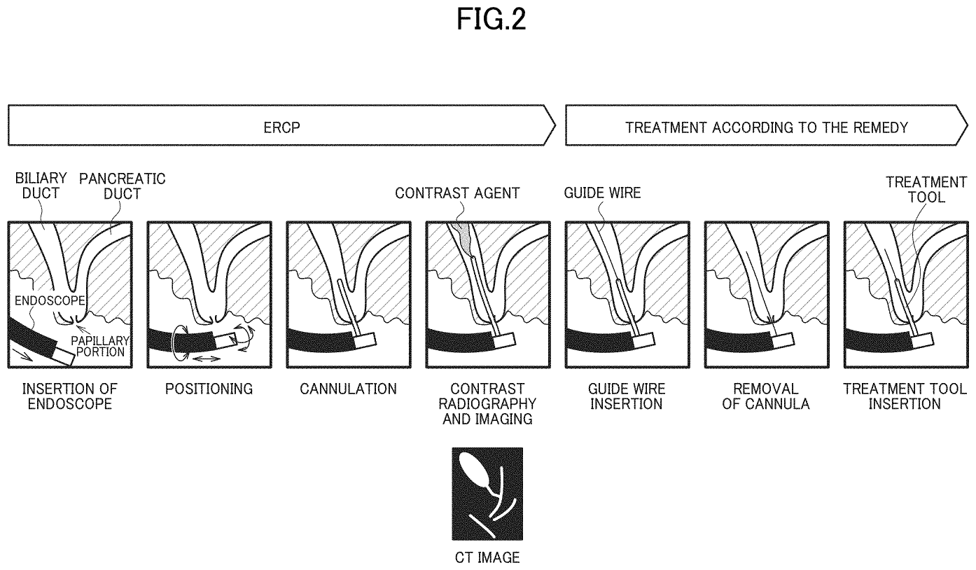

FIG. 2 shows a flow of the ERCP procedure. In ERCP, a side-viewing type endoscope in which a camera, an illumination lens, and an opening of a treatment tool channel are provided on a side surface of a distal end section of the endoscope is used. The camera is also referred to as an imaging device.

In the endoscope insertion step, the insertion section of the endoscope is inserted from the mouth to the duodenum through the esophagus and stomach. At this time, the insertion section is inserted until the papillary portion becomes roughly visible in the field of view of the endoscope. Next, in the positioning step, the position of the endoscope is adjusted relative to the papillary portion. Specifically, the position of the distal end section of the endoscope is adjusted so that the papillary portion is within the imaging range of the camera of the endoscope. Alternatively, the position of the distal end section of the endoscope is adjusted so that the camera of the endoscope is facing directly in front of the papillary portion and the papillary portion appears in the center of the field of view.

Then, in the cannulation step, a cannula is inserted from the papillary portion into the biliary duct. Specifically, the cannula is inserted into the treatment tool channel of the endoscope so that the cannula protrudes from the channel opening of the distal end section of the endoscope. The distal end of the cannula is inserted into the common duct from the opening of the common duct, and the cannula is further inserted through the confluence of the biliary duct and the pancreatic duct toward the direction of the biliary duct. Cannulation refers to insertion of a cannula into a body. A cannula is a medical tube that is inserted into a body for medical purposes.

Next, in the contrast radiography and imaging step, a contrast agent is injected into the cannula and ejected into the biliary duct from the distal end of the cannula. By performing X-ray or CT imaging in this state, an X-ray image or a CT (Computed Tomography) image showing the biliary duct, gallbladder, and pancreatic duct can be obtained. The procedure of ERCP has been described. After the procedure, various treatments are performed according to the results of diagnosis based on the X-ray image or CT image. An example of the treatment is described below.

In a guide wire insertion step, a guide wire is inserted into a cannula so that the guide wire is protruded from the distal end of the cannula, and the guide wire is inserted into the biliary duct. In a cannula removing step, the cannula is removed while leaving the guide wire inside the biliary duct. As a result, only the guide wire protrudes from the distal end section of the endoscope, indwelling in the biliary duct. Next, in a treatment tool insertion step, the treatment tool is inserted into the biliary duct along the guide wire. An example of a treatment tool is a basket or stent. The basket is used with a catheter. While allowing the guide wire to pass through the catheter, the catheter is inserted into the biliary duct along the guide wire. A basket made of a plurality of metal wires is inserted into the biliary duct from the distal end of the catheter, an object to be removed, such as a gallstone, is placed in the basket and held, and the object to be removed is taken out from the biliary duct by removing the basket and catheter in this state from the biliary duct. A stent can also be used in a similar manner with a catheter and inserted into the biliary duct from the distal end of the catheter. The narrow portion of the biliary duct can be widened by inserting a stent; further, by keeping the stent therein, the narrow portion can be held in a widened state by the indwelling stent.

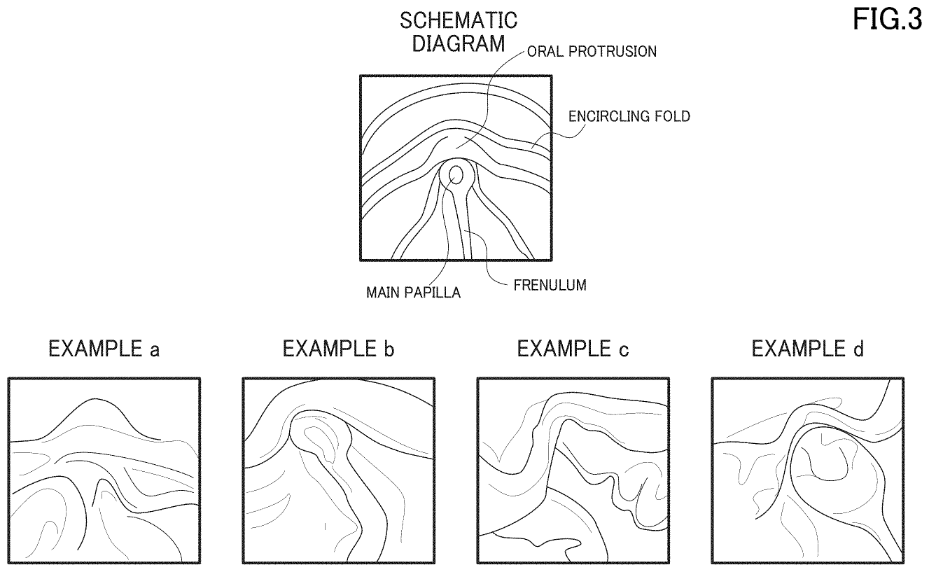

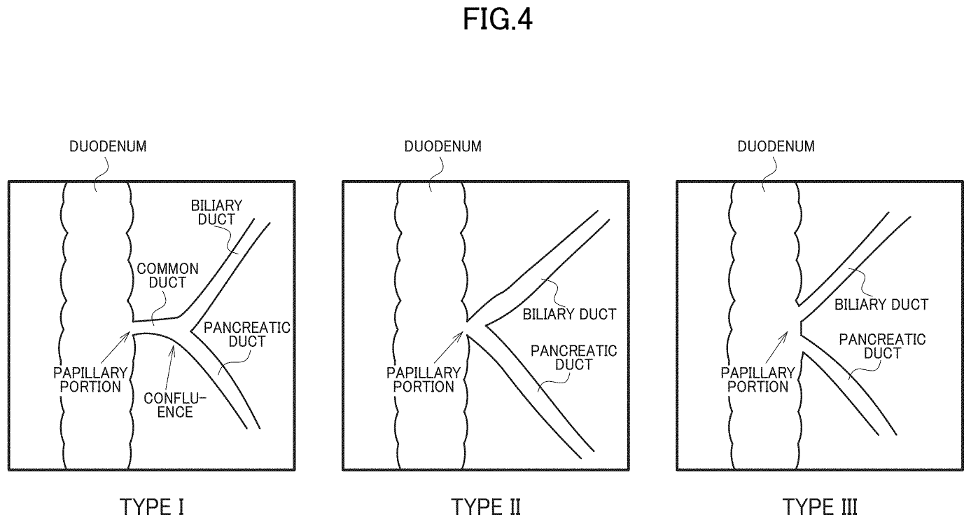

The ERCP procedure is performed as described above; however, in the positioning step, the procedure becomes difficult due to the individual differences in the papillary portions or luminal tissues. The following describes this issue with reference to FIGS. 3 and 4.

FIG. 3 is a diagram schematically showing the form of the papillary portion as viewed directly from the front thereof, and examples of individual differences of the papillary portion. As shown in the schematic diagram, structures peculiar to the papillary portion are present around the main papilla, which is the opening of the luminal tissue. Specifically, structures called frenulum, encircling fold, and oral protrusion can be present around the main papilla.

Although the schematic diagram shows a typical form of the papillary portion, as shown in Examples a to d, the form of the papillary portion varies between individuals and different for each patient. Examples of the differences include unclear main papilla, frenulum, encircling fold, or oral protrusion, and other forms significantly different from the typical form. In addition, the opening of the luminal tissue is often closed; in this case, it is difficult to precisely recognize the opening by visual observation.

FIG. 4 is a cross-sectional view showing the form of the luminal tissue and its opening. The forms of the luminal tissue and its opening are classified into Type I(Y-shaped), Type II(V-shaped), and Type III(U-shaped or separated). In Type I, a biliary duct and a pancreatic duct merge into a common duct at the confluence thereof, and the common duct opens to the papillary portion. In Type II, the biliary duct and the pancreatic duct open to the papillary portion at the confluence thereof, and there is no common duct. In Type III, the biliary duct and the pancreatic duct are separately open to the papillary portion and there are no confluence or common duct. Type I is most common, but there are also Type II and Type III patients.

When cannulation into the biliary duct is performed, it is basically performed by referring to an endoscope image showing the papillary portion, as shown in Examples a to d in FIG. 3. As described with reference to FIGS. 3 and 4, there are various forms of papillary portion and luminal tissue, and it is difficult to specify the insertion position and insertion direction of the cannula from the endoscope image.

On the other hand, the operator estimates the position of the opening and the travelling direction of the biliary duct based on past cases, experiences, and the like while viewing the endoscope image, and tries to insert the cannula from the opening into the biliary duct according to the estimation. At this time, in order to more accurately estimate the position of the opening and the travelling direction of the biliary duct, it is desirable that the position of the papillary portion in the image and the angle of view of the image are easy to compare with those in the past cases or are familiar to the operator.

As shown in FIG. 1, such positioning of the endoscope is performed by operating the distal end of the endoscope insertion section reaching the duodenum from outside the body. However, since the insertion section and the organ through which the insertion section passes are flexible, the operation performed at the base end of the insertion section is not easily transmitted to the distal end section. In addition, since the distal end section of the endoscope is not fixed to the duodenum and is free to move relative to the duodenum, the distal end section of the endoscope is not stable with respect to the papillary portion, and the positional relationship between the distal end section and the papillary portion is not easily determined. For these reasons, it is difficult to adjust the position of the distal end section of the endoscope so that the field of view of the endoscope is facing directly in front of the papillary portion or so that the papillary portion appears in the center of the field of view.

### Procedure Flow and Medical System According to the Present Embodiment

Therefore, in the present embodiment, the above-described positioning is automated by an electric medical system to assist the ERCP procedure. Further, by adding a configuration in which the insertion section of the endoscope is held in the duodenum, the electrically-driven force can be easily transmitted to the distal end section of the endoscope and the position of the distal end section can be desirably controlled. The details of this structure are described below.

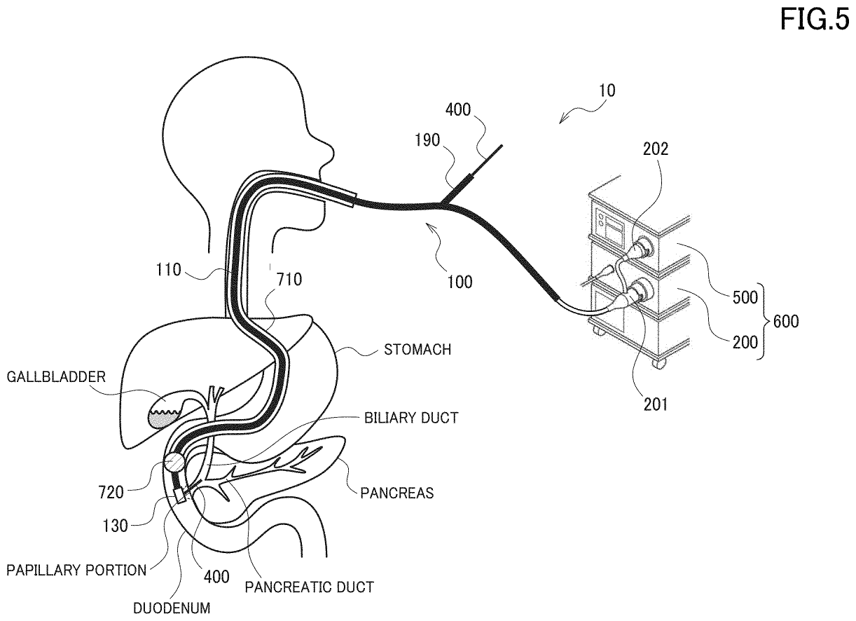

FIG. 5 shows a basic configuration example of a medical system 10 according to the present embodiment. The medical system 10 includes an endoscope 100, an overtube 710, a balloon 720, a treatment tool 400, and a control device 600. The medical system 10 is also referred to as an endoscope system or an electric endoscope system.

The overtube 710 is a tube with a variable hardness that covers the insertion section 110 of the endoscope 100. The balloon 720 is provided near the distal end on the outer side of the overtube 710. When the endoscope 100 and the overtube 710 are inserted into the body, at least the bending section of the insertion section 110 is exposed from the distal end of the overtube 710. The bending section refers to a section structured to be bent at an angle corresponding to the bending operation in the vicinity of the distal end of the insertion section 110. The base end of the overtube 710 is present outside the body. The base end side of the insertion section 110 is exposed from the base end of the overtube 710.

An insertion opening 190 of the treatment tool is provided at the base end side of the insertion section 110, and a treatment tool channel for allowing the treatment tool 400 to pass through from the insertion opening 190 to the opening of the distal end section 130 is provided inside the insertion section 110. The insertion opening 190 of the treatment tool is also called a forceps opening; however, the treatment tool to be used is not limited to forceps.

The endoscope 100 is detachably connected to a control device 600 using connectors 201 and 202. The control device 600 includes a drive control device 200 to which the connector 201 is connected, and a video control device 500 to which the connector 202 is connected. The drive control device 200 controls the electrical driving of the endoscope 100 via the connector 201. Although not shown in FIG. 5, an operation device for manually operating the electrical driving may be connected to the drive control device 200. The video control device 500 receives an image signal from a camera provided at the distal end section 130 of the endoscope 100 via the connector 202, generates a display image from the image signal, and displays it on a display device (not shown). In FIG. 5, the drive control device 200 and the video control device 500 are shown as separate devices, but they may be structured as a single device. In this case, the connectors 201 and 202 may be integrated into a single connector.

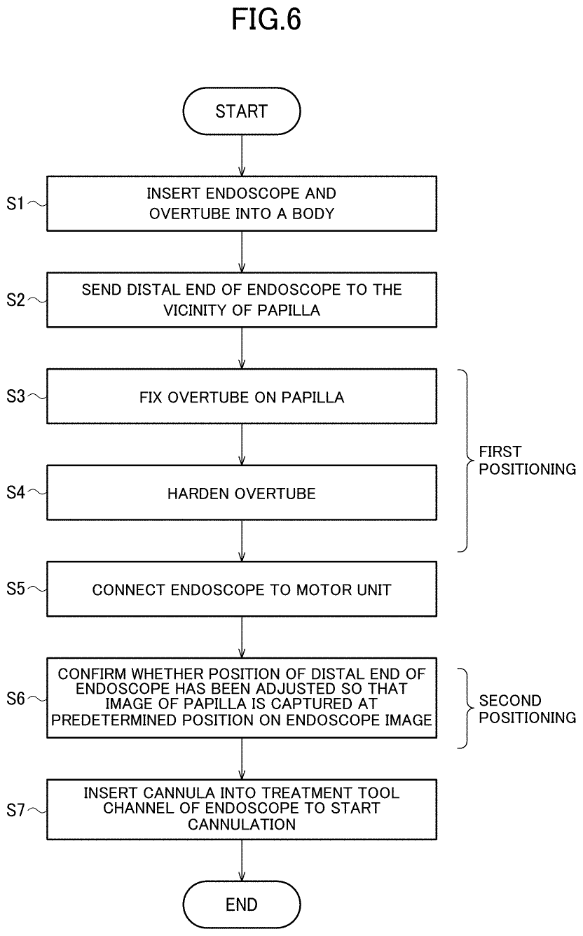

FIG. 6 shows a first flow of the procedure in the present embodiment. Here, an electric endoscope is assumed in which the forward and backward movement of the insertion section 110 of the endoscope 100, the bending of the bending section of the insertion section 110, and the rolling rotation of the insertion section 110 are electrically driven. However, it is sufficient that at least one of these functions is electrically driven. The term “electrical driving” means that the endoscope is driven by a motor or the like based on an electrical signal for controlling the endoscopic operation. For example, when the electrical driving is manually operated, an operation input to the operation device is converted into an electrical signal, and the endoscope is driven based on the electrical signal. In the following, the forward and backward movement may be simply referred to as “forward/backward movement”.

In step S1, the operator inserts the insertion section 110 of the endoscope 100 and the overtube 710 into the duodenum. More specifically, in a state where the insertion section 110 is inserted into the overtube, the insertion section 110 and the overtube 710 are inserted into the duodenum together. The overtube 710, which is changeable in hardness, is soft in step S1. For example, the operator can move the insertion section 110 and the overtube 710 forward by a non-electrically-driven manual operation so that they are inserted into the body. The non-electrical driving means that the endoscope 100 is not electrically driven by a motor or the like, instead, the force applied to the operation section is directly transmitted to the endoscope by a wire or the like, thereby operating the endoscope. For example, in the present embodiment, steps S1 to S4 are not electrically driven. In this case, it is sufficient that at least the forward/backward movement is not electrically driven, and the bending, the rolling rotation, or both may be manually operated by electrical driving.

In step S2, the operator inserts the insertion section 110 until the distal end section 130 reaches the vicinity of the papillary portion. For example, when the operator manually inserts the insertion section 110 by non-electrical driving, the operator inserts the insertion section 110 until the papillary portion becomes visible in the endoscope image. At this point, the distal end of the endoscope 100 does not need to accurately reach the papillary portion; the distal end of the endoscope 100 may reach a position before the papillary portion or past the papillary portion.

In step S3, the operator fixes the distal end of the overtube 710 to the duodenum. As an example, the operator performs an operation to inflate the balloon 720 provided near the distal end of the overtube 710, and fixes the distal end of the overtube 710 to the duodenum by the balloon 720. In step S4, the operator performs an operation to harden the overtube 710. At this time, the overtube 710 is hardened while maintaining its shape in a state immediately before hardening, that is, the shape when it is inserted from the mouth to the duodenum. As a result, the insertion section 110 is held by the hardened overtube 710 and the balloon 720, thereby fixing the insertion route of the insertion section 110. These steps S3 and S4 are referred to as first positioning.

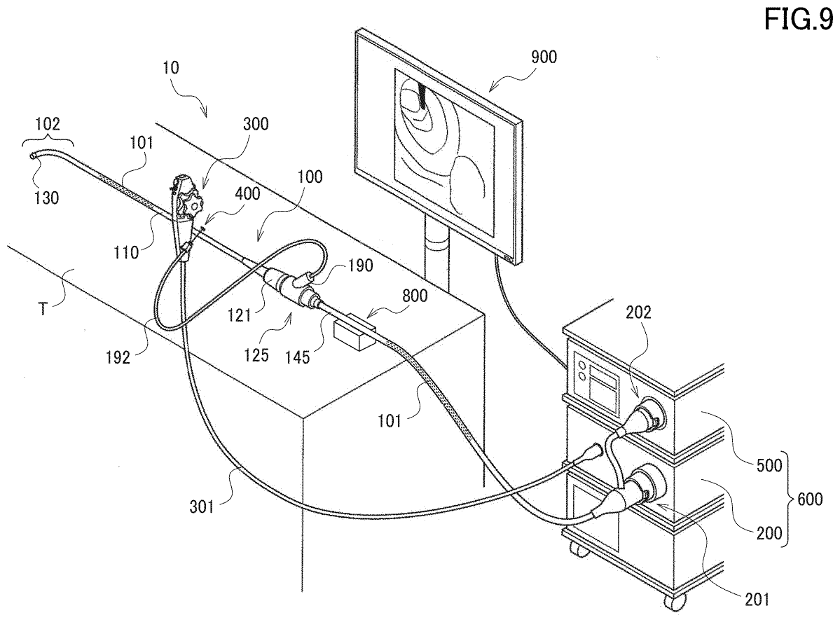

In step S5, the endoscope 100 is connected to the motor, and the non-electrical driving is switched to electrical driving. The method of switching between the non-electrical driving and the electrical driving varies depending on the configuration of the drive mechanism. For example, when the medical system 10, which is described later with reference to FIG. 9, is used, in steps 51 to S4, the forward/backward movement is non-electrically driven and the bending and the rolling rotation are electrically driven. In this case, the forward/backward movement may be switched from the non-electrical driving to the electrical driving by connecting the endoscope 100 to the forward/backward drive device 800. Further, when the bending operation by non-electrical driving is enabled by providing a bending operation dial or the like capable of non-electrically performing the bending operation, the bending movement may be switched from the non-electrical driving to the electrical driving, for example, by connecting the connector 201 to the drive control device 200. Alternatively, even if the motor is kept connected, the motor may be structured to be detachable by a clutch mechanism or the like, and the non-electrical driving may be switched to the electrical driving by the clutch mechanism. Step S5 may be performed before step S1. For example, when the forward/backward movement is manually operated by electrical driving, the endoscope 100 may be connected to the motor before step S1.

In step S6, the drive control device 200 automatically positions the distal end section 130 at the papillary portion, and the operator confirms that the position of the distal end section 130 has been adjusted so that the papillary portion is captured at a predetermined position on the endoscope image. The drive control device 200 acquires an endoscope image from the video control device 500 and performs positioning of the distal end section 130 of the endoscope 100 based on the endoscope image. More specifically, the drive control device 200 controls the forward/backward movement, bending, or rolling rotation by electrical driving so that the papillary portion is captured at a position registered in advance on the endoscope image. The position registered in advance is, for example, the center of the image. The positioning can be performed so that the opening of the luminal tissue is captured at a position registered in advance. Further, the drive control device 200 may perform electrical driving control based on the endoscope image so that the camera faces directly the front of the papillary portion or so that the papillary portion is captured at an appropriate angle of view. The drive control device 200 may also adjust the angle of view in imaging the papillary portion by controlling the diameter of the balloon 720 by electrical driving based on the endoscope image so that the distance between the camera and the papillary portion can be changed without changing the line-of-sight direction of the camera. This step S6 is referred to as second positioning.

In step S7, the operator inserts a cannula into the treatment tool channel through the insertion opening 190 to start cannulation into the biliary duct.

In FIG. 6, although the operation of the balloon in step S3 and the hardening of the overtube in step S4 are performed by non-electrical driving, they may be performed by electrical driving. In this case, the operator inputs an instruction from the operation device, and the drive control device 200 may inflate the balloon or harden the overtube by electrical driving using the instruction as a trigger. Alternatively, the drive control device 200 may perform an image recognition process for detecting the papillary portion from the endoscope image, and may automatically inflate the balloon or harden the overtube using the detection of the papillary portion from the endoscope image as a trigger.

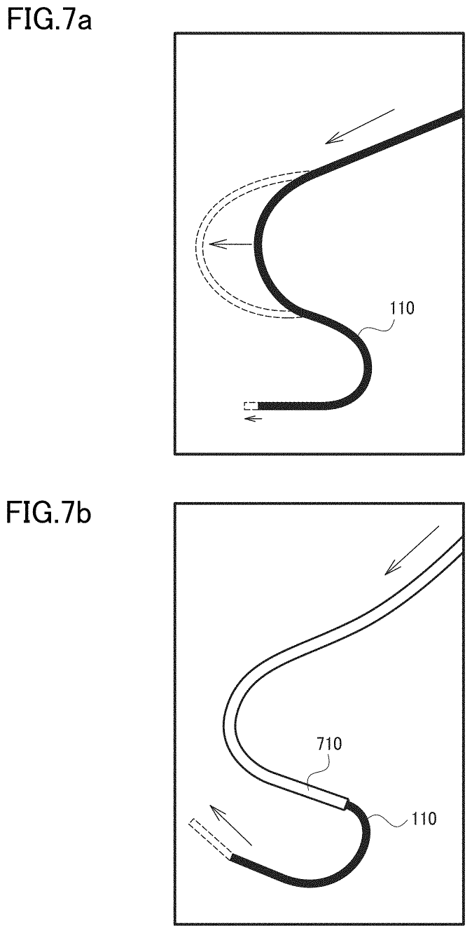

According to the procedure flow described above, by inflating the balloon 720 before hardening the overtube 710 in step S3, the position of the distal end of the overtube 710 does not shift when the overtube 710 is hardened. Specifically, the distal end of the overtube 710 can be accurately positioned. In addition, by the first positioning in steps S3 and S4, the insertion route of the insertion section 110 is held by the balloon 720 and the overtube 710. As a result, in the second positioning in step S6, the forward/backward movement, bending, or rolling rotation of the endoscope 100 due to the electrical driving is easily transmitted from the base end side to the distal end of the insertion section 110. This effect is described below with reference to FIGS. 7a and 7b.

FIGS. 7a and 7b show a comparison between a case without an overtube and a case with an overtube 710. The forward movement of the insertion section 110 is described herein as an example. The forward movement of the insertion section 110 is achieved by pushing the insertion section 110 in the axial direction by a slider mechanism or the like, which is described later. As shown in FIG. 7a, in the case where the insertion section 110 is not covered with the overtube 710, when the base end side of the insertion section 110 is pushed in the axial direction, the force is absorbed by the deformation of the insertion section 110 and is not easily transmitted to the distal end of the insertion section 110. This is because of the flexibility of the insertion section 110 and the stomach or the duodenum through which the insertion section 110 passes. As shown in FIG. 7b, in the case where the insertion section 110 is covered with the hardened overtube 710, when the base end side of the insertion section 110 is pushed in the axial direction, the insertion section 110 is moved forward in the overtube 710 using the hardened overtube 110 as a guide. As a result, the forward driving of the base end side is efficiently transmitted to the distal end section of the insertion section 110. Also, for the bending or rolling rotation, the electrical driving from the base end side is efficiently transmitted to the distal end section of the insertion section 110 by holding the insertion section 110 by the overtube 710 and the balloon 720.

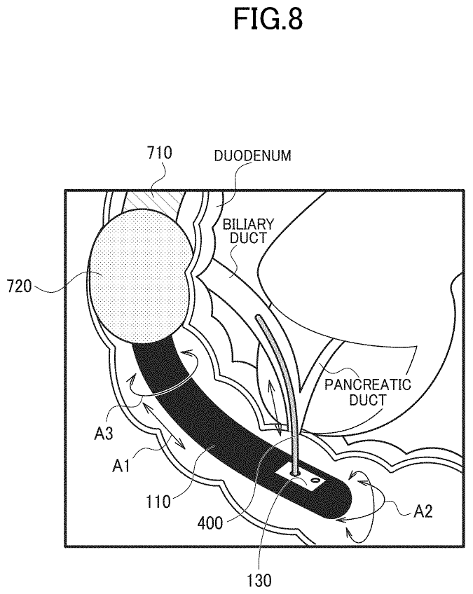

FIG. 8 shows the vicinity of the distal end of an endoscope positioned by the overtube 710 and the balloon 720. As shown in FIG. 8, the balloon 720 is fixed at a position slightly apart from the papillary portion to the pyloric side of the stomach. More specifically, the balloon 720 is positioned closer to the base end of the insertion section 110 than the base end of the bending section of the insertion section 110. By combining such a balloon 720 with the overtube 710 having a variable hardness, the bending section exposed to the papillary portion side from the balloon 720 and the distal end section 130 can be freely operated without being fixed, and the electrical driving from the base end side can be efficiently transmitted to the distal end section 130 of the endoscope.

The endoscopic operation by the electrical driving is the forward and backward movement shown as arrow A1, a bending movement shown as arrow A2, or a rolling rotation shown as arrow A3. The forward movement is a shift toward the distal end side along the axial direction of the insertion section 110, and the backward movement is a shift toward the base end side along the axial direction of the insertion section 110. The bending movement is a movement by which the angle of the distal end section 130 is changed due to the bending of the bending section. The bending movement includes bending movements in two orthogonal directions, which can be controlled independently. One of the two orthogonal directions is referred to as the vertical direction and the other is referred to as the horizontal direction. The rolling rotation is a rotation about an axis of the insertion section 110.

FIG. 8 shows an example in which the balloon 720 is attached to the distal end of the overtube 710 and the endoscope protrudes from the distal end of the overtube 710. However, it is sufficient that the overtube 710 and the balloon 720 are configured so that a portion of the bending section beyond the base end can freely move. For example, it may also be arranged such that a soft tube with a constant hardness extends beyond the overtube with a variable hardness, and the balloon 720 is attached to the boundary thereof. In this case, although a part of the base end side of the bending section is covered with the soft tube, its movement is not hindered.

Detailed Configuration Example of Medical System

FIG. 9 shows a detailed configuration example of the medical system 10. The medical system 10 is a system for observing or treating the inside of the body of a patient lying on an operating table T. The medical system 10 includes an endoscope 100, a control device 600, an operation device 300, a treatment tool 400, a forward/backward drive device 800, and a display device 900. The control device 600 includes a drive control device 200 and a video control device 500.

The endoscope 100 is a device to be inserted into a lumen of a patient for the observation of an affected part. In this embodiment, the side to be inserted into a lumen of a patient is referred to as “distal end side” and the side to be attached to the control device 600 is referred to as “base end side” or proximal end side. The endoscope 100 includes an insertion section 110, a connecting section 125, an extracorporeal soft section 145, and connectors 201 and 202. The insertion section 110, the connecting section 125, the extracorporeal soft section 145, and the connectors 201 and 202 are connected one another in this order from the distal end side.

The insertion section 110 is a portion to be inserted into a lumen of a patient, and is configured in a soft elongated shape. The insertion section 110 includes a bending section 102, an extracorporeal soft section for connecting the base end of the bending section 102 and the connecting section 125, and a distal end section 130 provided at the distal end of the bending section 102. An internal route 101 is provided inside the insertion section 110, the connecting section 125, and the extracorporeal soft section 145, and a bending wire passing through the internal route 101 is connected to the bending section 102. When the drive control device 200 drives the wire via the connector 201, the bending section 102 bends. Further, a raising base wire connected to a raising base provided at the distal end section 130 is connected to the connector 201 through the internal route 101. As the drive control device 200 drives the raising base wire, the raising angle of the treatment tool 400 protruding from the side surface of the distal end section 130 is changed. The side surface of the distal end section 130 is provided with a camera, an illumination lens, and an opening of a treatment tool channel. An image signal line for connecting the camera and the connector 202 is provided in the internal route 101, and an image signal is transmitted from the camera to the video control device 500 via the image signal line. The video control device 500 displays an endoscope image generated from the image signal on the display device 900.

The connecting section 125 is provided with an insertion opening 190 of the treatment tool and a rolling operation section 121. The treatment tool channel is provided in the internal route 101, one end of which is open to the distal end section 130 and the other end of which is open to the insertion opening 190 of the treatment tool. An extension tube 192 extending from the insertion opening 190 to the operation device 300 is connected to the insertion opening 190. The treatment tool 400 is inserted from an opening on the operation device 300 side of the extension tube 192, and protrudes to the opening of the distal end section 130 via the insertion opening 190 and the treatment tool channel. The extension tube 192 may be omitted, and the treatment tool 400 may be inserted directly through the insertion opening 190. The rolling operation section 121 is attached to the connecting section 125 so as to be rotatable about the axial direction of the insertion section 110. By rotating the rolling operation section 121, the insertion section 110 undergoes rolling rotation. As described later, the rolling operation section 121 can be electrically driven.

The forward/backward drive device 800 is a drive device for moving the insertion section 110 forward and backward by electrical driving. An extracorporeal soft section 145 is detachable from the forward/backward drive device 800, and an insertion section 110 moves forward and backward when the forward/backward drive device 800 causes the extracorporeal soft section 145 to slide in the axial direction in a state in which the extracorporeal soft section 145 is mounted on the forward/backward drive device 800. Although FIG. 9 shows an example in which the extracorporeal soft section 145 and the forward/backward drive device 800 are detachable, there is no such limitation, and it may be arranged such that the connecting section 125 and the forward/backward drive device 800 are detachable.

The operation device 300 is detachably connected to the drive control device 200 via an operation cable 301. The operation device 300 may communicate with the drive control device 200 through wireless communication instead of wired communication. When an operator operates the operation device 300, a signal of the operation input is transmitted to the drive control device 200 via the operation cable 301, and the drive control device 200 electrically drives the endoscope 100 to enable an endoscopic operation corresponding to the operation input based on the signal of the operation input. The operation device 300 has an operation input section having five or more channels corresponding to the forward and backward movement of the endoscope 100, the bending movements in two directions and the rolling rotation, and the operation of the raising base. If one or more of these operations are not electrically driven, the operation input section may be omitted. Each operation input section includes, for example, a dial, a joystick, a D-pad, a button, a switch, a touch panel, and the like.

The drive control device 200 electrically drives the endoscope 100 by driving a built-in motor based on an operation input to the operation device 300. Alternatively, when the motor is present outside the drive control device 200, the drive control device 200 transmits a control signal to the external motor based on an operation input to the operation device 300, thereby controlling the electrical driving. In addition, the drive control device 200 may drive a built-in pump or the like based on an operation input to the operation device 300, thereby causing the endoscope 100 to perform air supply and/or suction. The air supply and/or suction are performed through an air supply/suction tube provided in the internal route 101. One end of the air supply/suction tube opens to the distal end section 130 of the endoscope 100, while the other end is connected to the drive control device 200 via the connector 201. In addition, the treatment tool channel may be extended to the connector 201, and the treatment tool channel may also be used as an air supply/suction tube.

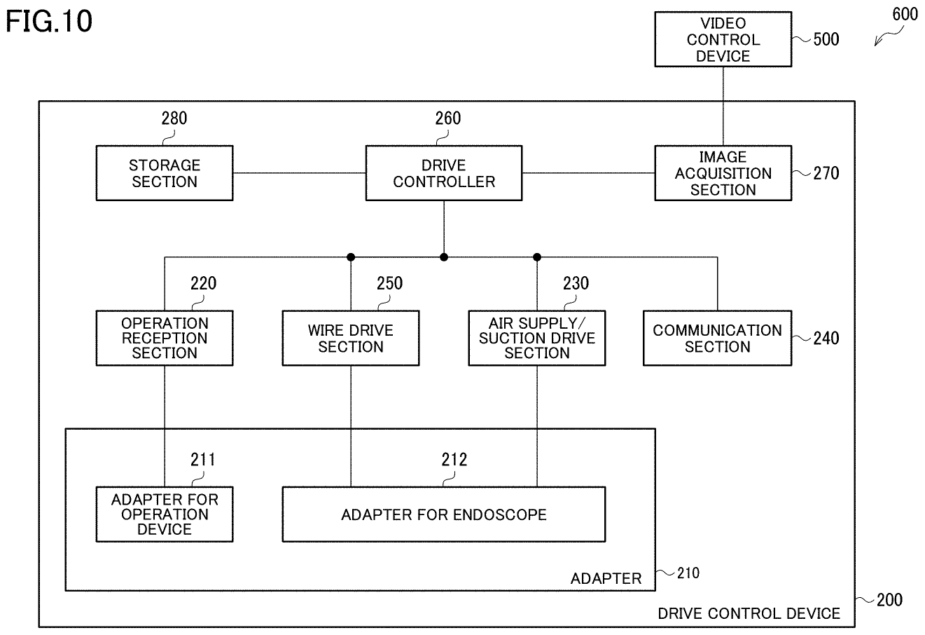

FIG. 10 shows a detailed configuration example of a drive control device 200. The drive control device 200 includes an image acquisition section 270, a storage section 280, a drive controller 260, an operation reception section 220, a wire drive section 250, an air supply/suction drive section 230, a communication section 240, and an adapter 210.

The adapter 210 includes an operation device adapter 211 to which the operation cable 301 is detachably connected, and an endoscope adapter 212 to which the connector 201 of the endoscope 100 is detachably connected.

The wire drive section 250 drives the bending movement of the bending section 102 of the endoscope 100 or the operation of the raising base of the treatment tool 400 based on the control signal from the drive controller 260. The wire drive section 250 includes a bending movement motor for driving the bending section 102 of the endoscope 100 and a raising base motor for driving the raising base. The endoscope adapter 212 has a bending movement coupling for enabling coupling to the bending wire on the endoscope 100 side. When the bending movement motor drives the coupling, the driving force is transmitted to the bending wire on the endoscope 100 side. Further, the endoscope adapter 212 has a raising base coupling for enabling coupling to the raising base wire on the endoscope 100 side. When the raising base motor drives the coupling mechanism, the driving force is transmitted to the raising base wire on the endoscope 100 side.

The air supply/suction drive section 230 drives air supply and/or suction of the endoscope 100 based on a control signal from the drive controller 260. The air supply/suction drive section 230 is connected to an air supply/suction tube of the endoscope 100 via the endoscope adapter 212. The air supply/suction drive section 230 includes a pump or the like, and supplies air to the air supply/suction tube or sucks air from the air supply/suction tube 172.

The communication section 240 (e.g., transceiver) communicates with a drive device provided outside the drive control device 200. The communication may be wireless communication or wired communication. The drive device provided outside is a forward/backward drive device 800 for performing forward and backward movement, a rolling drive device for performing the rolling rotation, an overtube drive device for changing the hardness of the overtube 710, a balloon drive device for changing the diameter of the balloon 720 or the like.

The drive controller 260 controls the forward and backward movement, the bending movement and the rolling rotation of the endoscope 100, the raising angle of the treatment tool 400 made by the raising base, and the air supply and/or suction by the endoscope 100. When the hardness control of the overtube 710 or the diameter control of the balloon 720 is electrically driven, the drive controller 260 performs the control thereof. The drive controller 260 is, for example, a processor such as a CPU (Central Processing Unit), a GPU (Graphics Processing Unit), a DSP (Digital Signal Processor), or the like. For example, the storage section 280 stores a computer-readable program, and the functions of the drive controller 260 are implemented as processes as the processor executes the program. However, the hardware of the drive controller 260 is not limited to that described above, and may be structured using circuits with various configurations.

The electric control performed by the drive controller 260 includes a manual mode in which the operator manually operates the electrical driving of the endoscope 100 or the like and an automatic control mode in which the electrical driving of the endoscope 100 or the like is automatically controlled based on an endoscope image. In the automatic mode of the present embodiment, the positioning step described with reference to FIG. 2 is automated. In the automatic mode, at least one of the forward and backward movement, the bending movement, and the rolling rotation of the endoscope 100 may be automated. That is, the raising angle of the treatment tool 400 by the raising base, the hardness control of the overtube 710, the diameter control of the balloon 720, the air supply and/or suction of the endoscope 100, or a part of the forward and backward movement, the bending movement or rolling rotation of the endoscope 100 may be manually operated.

First, the manual mode is described below. The operation reception section 220 receives an operation input signal from the operation device 300 via the operation cable 301 attached to the operation device adapter 221. When the operation device 300 communicates with the drive control device 200 by wireless communication, the operation reception section 220 may be a wireless communication circuit.

The drive controller 260 controls the electrical driving based on an operation input signal from the operation reception section 220. Specifically, when the bending operation is performed, the drive controller 260 outputs a control signal indicating the bending direction or the bending angle to the wire drive section 250, and the wire drive section 250 drives the bending wire so that the bending section 102 bends in the bending direction or the bending angle. Also, when the forward and backward movement operation is performed, the drive controller 260 transmits a control signal indicating the forward/backward direction or the forward/backward movement amount to the forward/backward drive device via the communication section 240, and the forward/backward drive device moves the extracorporeal soft section 140 forward or backward so that the endoscope 100 moves forward or backward in the forward/backward direction or the forward/backward movement amount. Further, when the rolling rotation operation is performed, the drive controller 260 transmits a control signal indicating the rolling rotation direction or the rolling rotation angle to the rolling drive device via the communication section 240, and the rolling drive device performs rolling rotation of the insertion section 110 so that the endoscope 100 undergoes rolling rotation in the rolling rotation direction or at the rolling rotation angle. Similar controls are performed for other electrical driving.

Next, the automatic control mode is described below. The image acquisition section 270 is a communication interface for receiving image data of an endoscope image from the video control device 500 by wired communication or wireless communication. The image acquisition section 270 outputs image data of the received endoscope image to the drive controller 260.

The storage section 280 stores a reference image of the papillary portion that serves as a reference for positioning with respect to the papillary portion. The reference image is an image in which the papillary portion is captured such that the opening of the luminal tissue appears at a predetermined position. The predetermined position is, for example, the center of the image, and corresponds to the “position registered in advance” described above. The storage section 280 may store a plurality of reference images corresponding to the various forms described with reference to FIG. 3 or FIG. 4. The storage section 280 is a storage device such as a semiconductor memory or a magnetic storage device. The semiconductor memory may be a volatile memory such as a SRAM or a DRAM, or a nonvolatile memory such as an EEPROM.

The drive controller 260 controls the endoscopic operation or the diameter of the balloon 720 so that the position of the papillary portion in the endoscope image is brought close to the position of the papillary portion in the reference image. For example, the drive controller 260 extracts an image feature amount of the papillary portion from the endoscope image and the reference image, and determines the position of the papillary portion in the endoscope image based on the comparison result of the image feature amount. The drive controller 260 can control the endoscopic operation or the diameter of the balloon 720 so that the position of the opening of the luminal tissue on the endoscope image comes close to a predetermined position. For example, a reference image in which the opening of the luminal tissue is located at a predetermined position is stored in the storage section 280. The drive controller 260 performs positioning of the opening of the luminal tissue by performing positioning of the papillary portion. Even in a case where the opening of the luminal tissue is closed and the opening cannot be recognized from the image, it is possible to perform the positioning so that the opening is present at a predetermined position in the image by performing positioning of the papillary portion. The storage section 280 may store the image feature amount extracted from the reference image, instead of the reference image.

The drive controller 260 may also control the endoscopic operation or the raising angle of the treatment tool 400 so that the distal end of the treatment tool 400 is directed toward the opening of the luminal tissue based on the endoscope image. Alternatively, the drive controller 260 may also control the endoscopic operation or the raising angle of the treatment tool 400 so that the distal end of the treatment tool 400 is directed toward the travelling direction of the biliary duct based on the endoscope image. For example, it may be arranged such that information on the travelling direction of the biliary duct is given to the reference image, and the distal end of the treatment tool 400 is controlled to be directed to the travelling direction of the biliary duct based on the information. Here, the travelling direction is a two dimensional direction on the endoscope image. That is, the endoscopic operation or the raising angle of the treatment tool 400 is controlled so that the travelling direction of the biliary duct and the direction to which the treatment tool 400 faces are substantially parallel to each other on the endoscope image. However, in a case where three dimensional information of the travelling direction of the biliary duct can be obtained from a CT image or the like, the control may be performed so that the travelling direction of the biliary duct and the direction to which the treatment tool 400 faces are substantially three dimensionally parallel.

Alternatively, the drive controller 260 may control the position of the opening of the luminal tissue in the endoscope image to be equal to the position registered in advance based on the result of the image recognition process using machine learning. More specifically, the storage section 280 stores a trained model, and the drive controller 260 performs the positioning control described above by performing a process based on the trained model. For example, the trained model receives input of an endoscope image and is trained to output information such as an endoscopic operation so that the position of the opening of the luminal tissue is located at a predetermined position in the endoscope image. Further, the trained model may be trained such that it receives input of an endoscope image and outputs information of the travelling direction of the biliary duct from the endoscope image. The drive controller 260 presumes information on an endoscopic operation or the like from an endoscope image by a process based on the trained model, and outputs a control signal for controlling the endoscopic operation or the raising angle of the treatment tool or the like to the wire drive section 250 or the like based on the information. In this example, the storage section 280 does not need to store the reference image or the image feature amount, and the “position registered in advance” of the opening of the luminal tissue is already reflected in the trained model by learning.

As described above, when cannulation into the biliary duct is performed, it is difficult to estimate the insertion position and the insertion direction of the cannula from the endoscope image of the papillary portion. In this regard, according to the present embodiment described above, in the second positioning after the first positioning by the overtube 710 or the like, the electrical driving of the endoscopic operation or the like is automatically controlled based on the endoscope image, so that the distal end section 130 of the endoscope 100 is automatically positioned with respect to papillary portion. As a result, it is not necessary to perform minute position adjustment by non-electric manual operation, and it is possible to assist the ERCP procedure performed by an inexperienced operator or the like. In addition, since the position of the papillary portion in the endoscope image is automatically controlled so as to be located at the position registered in advance by the automatic control, the operator can easily specify the insertion position and the insertion direction of the cannula from the endoscope image. For example, since the opening of the luminal tissue is automatically controlled so as to be at a predetermined position in the image, the operator can more easily grasp the position of the opening even when the opening cannot be visually recognized from the image.

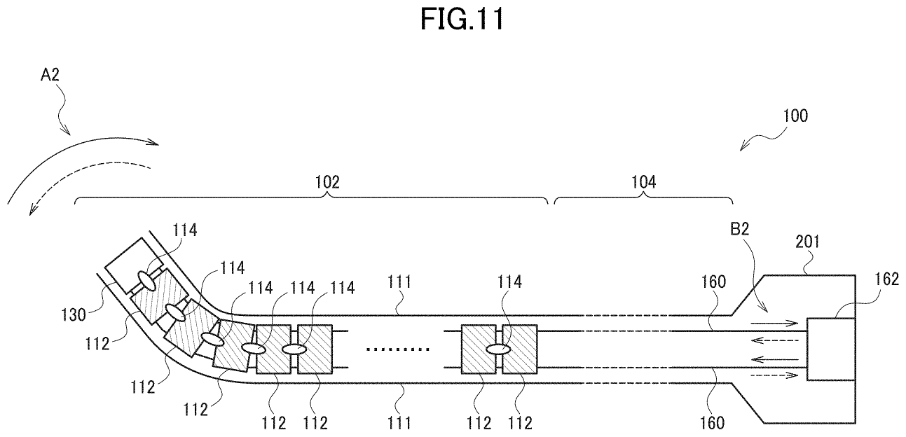

Detailed Configuration Example of Each Part of Medical System FIG. 11 is a schematic view of an endoscope 100 including a bending section 102 and a driving mechanism thereof An endoscope 100 includes a bending section 102, a soft section 104, and a connector 201. The soft section 104 corresponds to the extracorporeal soft section 145 described above with reference to FIG. 9. In FIG. 11, the connecting section 125 is omitted.

The bending section 102 and the soft section 104 are covered with an outer sheath 111. The inside of the tube of the outer sheath 111 corresponds to the internal route 101 in FIG. 9. The bending section 102 includes a plurality of bending pieces 112 and a distal end section 130 connected to the distal end of the bending pieces 112. Each of the plurality of bending pieces 112 and the distal end section 130 is connected in series from the base end side to the distal end side by a rotatable connecting section 114, thereby forming a multi joint structure. The connector 201 is provided with a coupling mechanism 162 on the endoscope side connected to a coupling mechanism on the drive control device 200 side. By attaching the connector 201 to the drive control device 200, it is possible to electrically drive the bending movement. A bending wire 160 is provided in the outer sheath 111. One end of the bending wire 160 is connected to the distal end section 130. The bending wire 160 passes through the soft section 104 by penetrating through a plurality of bending pieces 112, turns back in a coupling mechanism 162, passes through the soft section 104 again, penetrates through the plurality of bending pieces 112. The other end of the bending wire 160 is connected to the distal end section 130. The driving force from the wire drive section 250 is transmitted to the bending wire 160 via the coupling mechanism 162 as the pulling force of the bending wire 160.

As shown by the solid line arrow B2, when the upper wire in the figure is pulled, the lower wire is pushed, whereby the multiple joints of the bending pieces 112 are bent upward in the figure. As a result, as indicated by the solid line arrow A2, the bending section 102 is bent upward in the figure. When the lower wire in the figure is pulled as indicated by the dotted arrow B2, similarly, the bending section 102 is bent downward in the figure as indicated by the dotted arrow A2. As described with reference to FIG. 8, the bending section 102 can be bent independently in two orthogonal directions. Although FIG. 11 shows a bending mechanism for one direction, two sets of bending wires can be provided, and each bending wire can be bent independently in two directions by being pulled independently by the coupling mechanism 162.

Note that the mechanism for the electrically-driven bending is not limited to that described above. For example, a motor may be provided instead of the coupling mechanism 162. Specifically, it may be arranged such that the drive control device 200 transmits a control signal to the motor via the connector 201, and the motor drives the bending movement by pulling or relaxing the bending wire 160 based on the control signal.

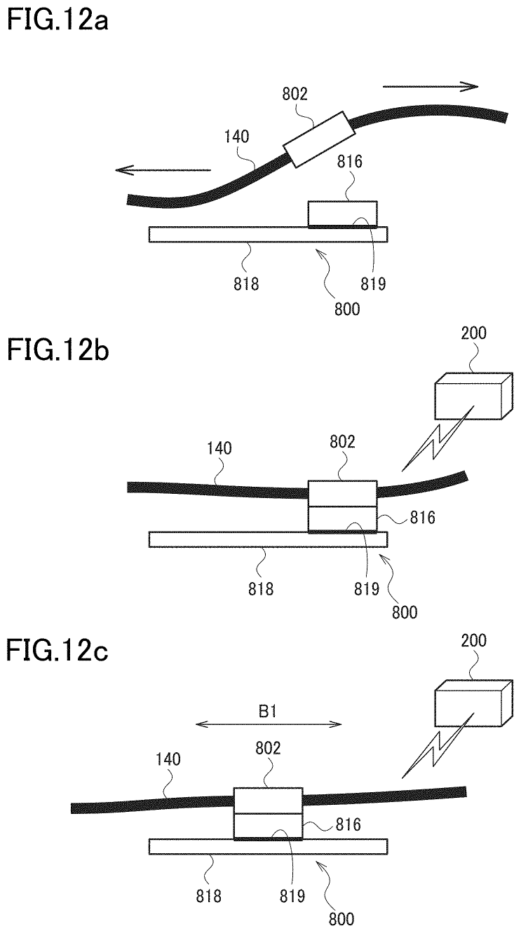

FIGS. 12a-12c show a detailed configuration example of a forward/backward drive device 800. The forward/backward drive device 800 includes a motor 816, a base 818, and a slider 819.

As shown in FIGS. 12a and 12b, the extracorporeal soft section 140 of the endoscope 100 is provided with an attachment 802 detachable from the motor 816. As shown in FIG. 12b, the attachment of the attachment 802 to the motor 816 enables electrical driving of forward/backward movement. As shown in FIG. 12c, the slider 819 supports the motor 816 while enabling the motor 816 to move linearly with respect to the base 818. The slider 819 is fixed to the operating table T shown in FIG. 9. As shown by arrow B1, the drive control device 200 transmits a forward or backward control signal to the motor 816 by wireless communication, and the motor 816 and the attachment 802 move linearly on the slider 819 based on the control signal. As a result, the forward and backward movement of the endoscope 100 shown by arrow A1 in FIG. 8 is achieved. Note that the drive control device 200 and the motor 816 may be connected by wired connection.

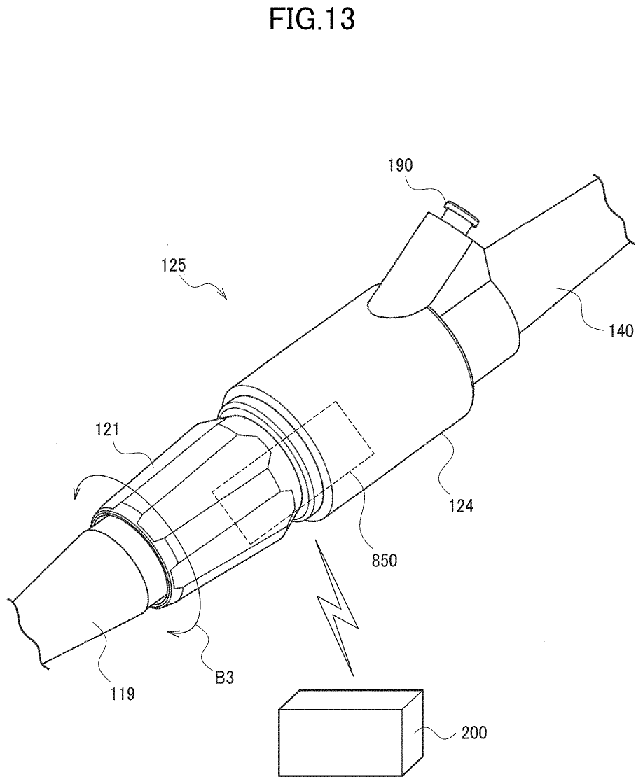

FIG. 13 is a perspective view of the connecting section 125 including a rolling drive device 850. The connecting section 125 includes a connecting section main body 124 and a rolling drive device 850.

The insertion opening 190 of the treatment tool is provided in the connecting section main body 124 and is connected to the treatment tool channel inside the connecting section main body 124. The connecting section main body 124 has a cylindrical shape, and a cylindrical member coaxial with the cylinder is rotatably provided inside the connecting section main body 124. The base end section of the intracorporeal soft section 145 is fixed to the outside of the cylindrical member, and the base end section serves as a rolling operation section 121. As a result, the intracorporeal soft section 145 and the cylindrical member can rotate with respect to the connecting section main body 124 about the axial direction of the intracorporeal soft section 145. The rolling drive device 850 is a motor provided inside the connecting section main body 124. As shown by arrow B3, the drive control device 200 transmits a rolling rotation control signal to the rolling drive device 850 by wireless communication, and the rolling drive device 850 rotates the base end section of the intracorporeal soft section 145 with respect to the connecting section main body 124 based on the control signal, thereby causing rolling rotation of the intracorporeal soft section 119 (145?). As a result, the rolling rotation of the endoscope 100 shown by arrow A3 in FIG. 8 is achieved. The rolling drive device 850 may include a clutch mechanism, and the rolling rotation may be switched between non-electrical driving and electrical driving by the clutch mechanism. The drive control device 200 and the rolling drive device 850 may be connected by wired connection via a signal line passing through the internal route 101.

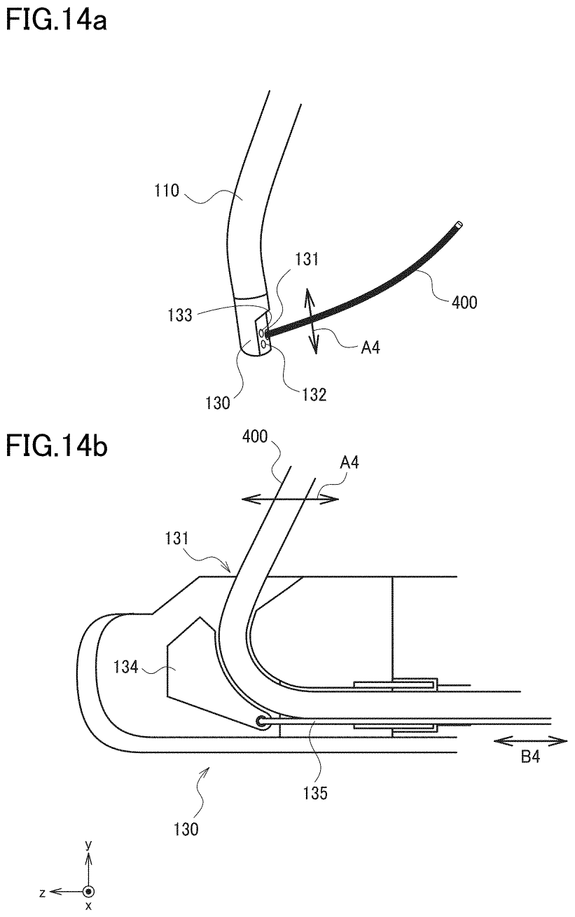

FIGS. 14a and 14b show a detailed configuration example of a distal end section 130 of an endoscope including a raising base of a treatment tool. FIG. 14a shows an external view of the distal end section 130. An opening 131 of a treatment tool channel, a camera 132, and an illumination lens 133 are provided on the side surface of the distal end section 130. As shown in FIG. 14B, the direction parallel to the axial direction of the distal end section 130 is defined as z direction, the direction parallel to the line-of-sight direction of the camera 132 is defined as y direction, and the direction orthogonal to the z direction and they direction is defined as x direction. FIG. 14b shows a cross-sectional view of the distal end section 130 in a plane that is parallel to the yz plane of the treatment tool channel and that passes through the opening 131 of the treatment tool channel.

The distal end section 130 includes a raising base 134 and a raising base wire 135. The raising base 134 is swingable about an axis parallel to the x direction. One end of the raising base wire 135 is connected to the raising base 134, while the other end is connected to the drive control device 200 via the connector 201. As shown by arrow B4, the wire drive section 250 of the drive control device 200 pushes and pulls the raising base wire 135 to swing the raising base 134, thereby, as shown by arrow A4, changing the raising angle of the treatment tool 400. The raising angle is an angle of the treatment tool 400 protruding from the opening 131. The raising angle can be defined, for example, by an angle formed by the treatment tool 400 protruding from the opening 131 and the z direction.

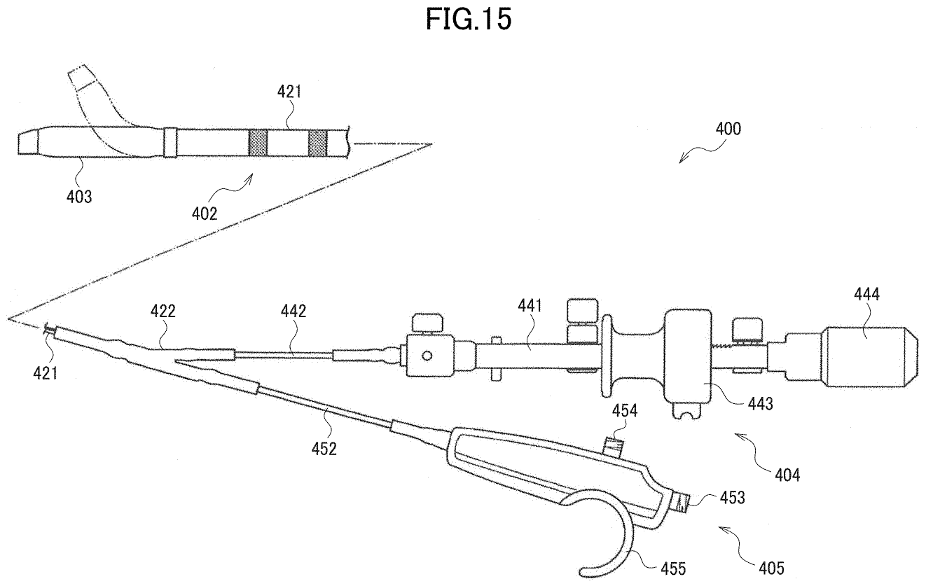

FIG. 15 shows a detailed configuration example of the treatment tool 400. Herein, as an example of the treatment tool 400, a cannula capable of operating bending of the distal end is shown. The treatment tool 400 includes a long-length insertion section 402 extending in the axial direction, a bending section 403 capable of bending movement, a first operation section 404 for operating the bending section 403, and a second operation section 405 for inserting a contrast agent or a guide wire.

The insertion section 402 has a tube 421, and the bending section 403 is connected to the distal end of the tube 421. In FIG. 15, the distal end side of the tube 421 is enlarged. The tube 421 is also referred to as a sheath. The operator holds the tube 421 of the treatment tool 400 inserted into the treatment tool channel of the endoscope 100, and pushes and pulls the tube 421 to move the treatment tool 400 forward and backward.

A connector 422 is connected to the base end of the tube 421. The first operation section 404 and the second operation section 405 are connected to the connector 422. The first operation section 404 includes a connecting tube 442, one end of which is connected to the connector 422, a first operation main body 441 connected to the other end of the connecting tube 442, a grip 444 fixed to the base end of the first operation main body 441, and a slider 443 provided movably forward and backward in the axial direction of the first operation main body 441. Inside the tube 421, the connector 422, the connecting tube 442, and the first operation main body 441, a wire for connecting the bending section 403 and the slider 443 is provided. When the operator pulls the slider 443 while holding the grip 444, the wire is pulled and the bending section 403 is bent.

The second operation section 405 includes a connecting tube 452, one end of which is connected to the connector 422, a second operation main body 451 connected to the other end of the connecting tube 452, a first opening 453 opened in the axial direction of the connecting tube 452 on the base end side of the second operation main body, a second opening 454 opened to the outer surface of the second operation main body 451, and a hook 455 provided on the second operation main body 451. The hook 455 has elasticity and is formed in a substantially C-shape, and is used for locking the treatment tool 400 to the endoscope 100 or the like. The first opening 453 and the second opening 454 are connected to the tube 421 via the second operation main body 451, the connecting tube 452, and the connector 422. By inserting a contrast agent or a guide wire from the first opening 453 or the second opening 454, the contrast agent can be injected into the body or the guide wire can be inserted into the body from the distal end of the treatment tool 400.

Although an example in which the treatment tool 400 is manually operated by non-electrical driving has been described herein, the operation of the treatment tool 400 may be operated by electrical driving. For example, using a method similar to the electrical driving of the endoscope 100, it is possible to perform the forward/backward movement of the treatment tool 400, the bending of the distal end, or the rolling rotation by electrical driving.

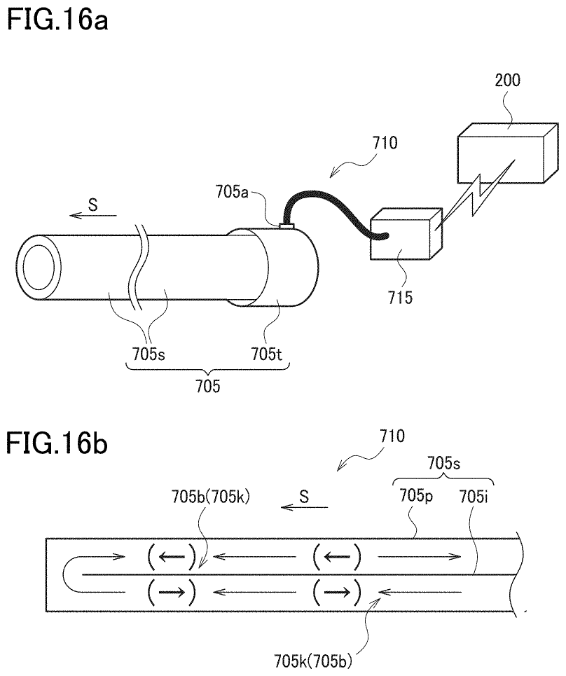

FIGS. 16a and 16b show a configuration example of the drive system 701 of the overtube 710. As shown in FIG. 16a, the drive system 701 includes an overtube 710 and an overtube drive device 715.

The overtube 710 is structured such that its hardness is variable, its shape is variable during softening, and its shape is maintained during hardening. Although an example of using a shape memory polymer with its hardness changeable according to the temperature is shown herein, the method of varying the hardness is not limited to this example, and, for example, a structure in which the hardness is varied using a multi joint structure made of a plurality of bridge members connected in series may be used. As shown in FIG. 16a, the overtube 710 includes an insertion section 705s and an operation section 705t. The operation section 705t is provided with a connecting section 705a, and the overtube drive device 715 is connected via the connecting section 705a.

FIG. 16b shows a cross-sectional view of the insertion section 705s in a cross section parallel to the insertion direction S. Although there are two tube walls in the cross section, the figure shows only one of them. The other wall has a similar structure. The insertion section 705s includes a tube member 705p and a shape memory polymer tube 705i.

The shape memory polymer tube 705i is hardened when a fluid having a temperature lower than the glass transition temperature is supplied, and is softened when a fluid having a temperature higher than the glass transition temperature is supplied. The shape memory polymer tube 705i is covered with the tube member 705p, and a supply path 705k and a collection path 705b connected to the distal end of the supply path 705k are provided between the shape memory polymer tube 705i and the inner wall of the tube member 705p. The overtube drive device 715 is a fluid supply device including a pump and the like. The fluid supply device supplies a fluid of a predetermined temperature to the supply path 705k and collects the fluid from the collection path 705b. The drive control device 200 transmits a control signal for controlling the hardness of the overtube 710 to the overtube drive device 715 by wireless communication, and the overtube drive device 715 changes the hardness of the overtube 710 by setting the temperature of the fluid based on the control signal. In FIG. 16b, in the overtube 710, the supply path 705k is located in the inner side and the collection path 705b is located in the outer side; however, the supply path 705k may be located in the outer side and the collection path 705b may be located in the inner side. Further, the drive control device 200 and the overtube drive device 715 may be connected by wired connection.

The mechanism for changing the hardness of the overtube by electrical driving is not limited to that described above. For example, it may be arranged such that a plurality of bridge members are connected in series, and the degree of contact between the bridge members are changed by electrical driving. Specifically, it may be arranged such that the overtube is softened by arranging adjacent bridge members so that they can slide and the overtube is hardened by arranging adjacent bridge members so that they are in contact with each other and cannot easily slide.

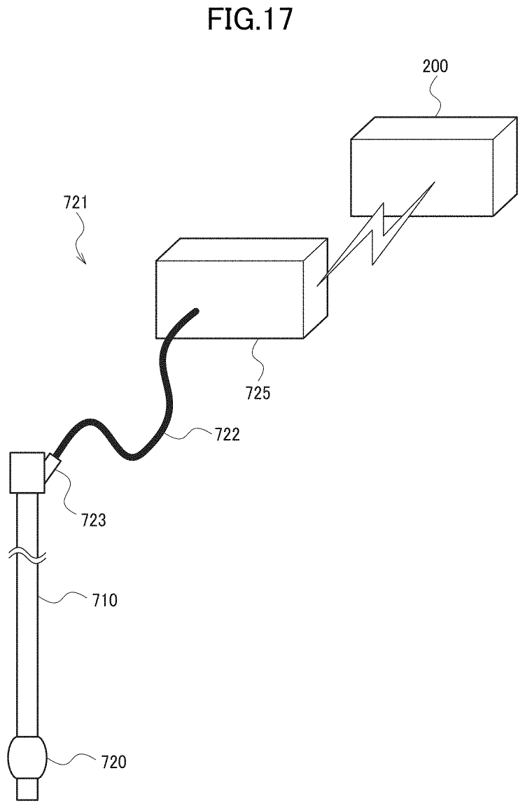

FIG. 17 shows a configuration example of the drive system 721 of the balloon 720. The drive system 721 includes a balloon 720, a connecting tube 722, and a balloon drive device 725.

The balloon 720 is made of an expandable member and is provided near the distal end of the overtube 710. The balloon 720 has a doughnut shape and is disposed so as to surround the outer periphery of the distal end section of the overtube 710. A balloon vent hole 723 is provided at the base end of the overtube 710, and the balloon vent hole 723 and the balloon 720 are connected to each other via a pipeline (not shown). The balloon vent hole 723 and the balloon drive device 725 are connected to each other by a connecting tube 722. The balloon drive device 725 includes a pump or the like, and inflates the balloon 720 by sending air to the balloon 720 through the connecting tube 722, or deflates the balloon 720 by sucking air from the balloon 720. The drive control device 200 transmits a control signal for controlling the diameter of the balloon 720 to the balloon drive device 725 by wireless communication, and the balloon drive device 725 inflates or deflates the balloon 720 based on the control signal, thereby controlling the diameter of the balloon 720. The drive control device 200 and the balloon drive device 725 may be connected by wired connection.

Modifications

Some modifications are described below. Each modification can be combined with any of the embodiments described above.

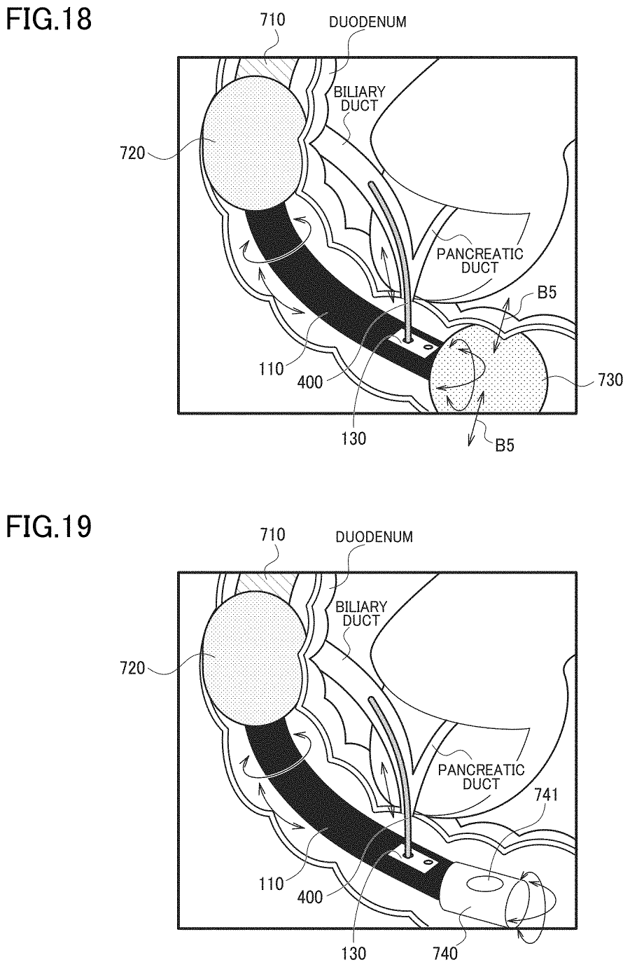

FIG. 18 shows a first modification of the holding member. In this modification, a balloon 730 is provided at the distal end section 130 of the endoscope 100. The diameter of the balloon 730 is variable by inflation and deflation. The diameter of the balloon 730 may be non-electrically controlled, or the diameter of the balloon 730 may be electrically controlled in a manner similar to that for the balloon 720. The electrical driving may be manual operation or automatic control based on an endoscope image. By fixing the distal end section 130 of the endoscope 100 to the duodenum by the balloon 730, the positional relationship between the distal end section 130 and the papillary portion can be stabilized during cannulation. As shown by arrow B5, by adjusting the diameter of the balloon 730, it is possible to adjust the distance between the distal end section 130 and the papillary portion without changing the angle of the distal end section 130.

FIG. 19 shows a second modification of the holding member. In this modification, a suction cap 740 is provided at the distal end section 130 of the endoscope 100. A suction opening 741 is provided on the side surface of the suction cap 740 in the same direction as the line-of-sight direction of the camera or the like. By sucking air through the suction opening 741, the intestinal wall of the duodenum is drawn toward the distal end section 130. This allows adjustment of the distance between the distal end section 130 and the papillary portion. This suction may be performed by non-electrical driving or electrical driving. The electrical driving may be manual operation or automatic control based on an endoscope image.

As a third modification of the holding member, a basket may be used instead of the balloon 720 or 730. By expanding or contracting the basket made of a plurality of bundled wires, a function similar to that of the balloon is achieved. As a fourth modification of the holding member, a grasper or a retractor may be provided at the distal end section 130 of the endoscope 100. The grasper or the retractor grasps the intestinal wall of the duodenum and pushes or pulls the intestinal wall. As a result, the distal end section 130 is held in the duodenum, and the distance between the distal end section 130 and the papillary portion is adjusted.

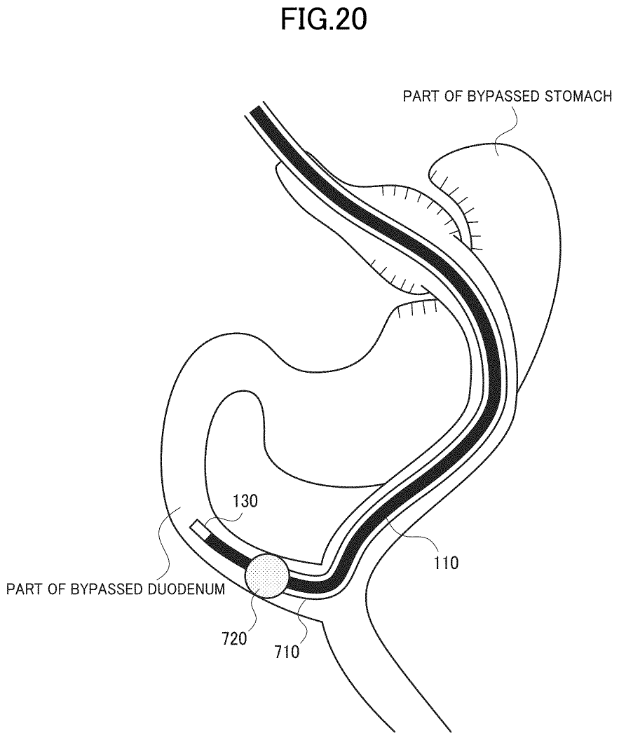

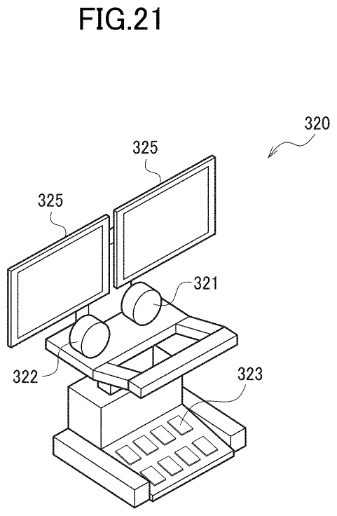

Next, a description is given for a modification using endoscopes or the like having various lengths corresponding to individual differences of patients. Since each patient has a different organ structure, it is difficult to use the same endoscope, overtube, or attachment for all patients. FIG. 20 shows an organ structure of a patient who had a gastric bypass surgery according to the Roux-en Y method, as an example of individual differences in organ structures. The Roux-en Y method is performed such that the stomach is divided into a bag connected to the esophagus and a bag connected to the duodenum, the ileum is connected to the bag connected to the esophagus, and the duodenum is connected in the middle of the ileum. The insertion distance from the mouth to the papillary portion differs between patients who had such a surgery and those who did not.

Therefore, by making the endoscope or overtube to be inserted into the body selectable for each patient by semi-reuse, single-use, or extension using attachment. Examples of reusable portions include the control device, the motor, the air supply/suction device, and the like. Examples of semi-reusable, single-use, or attachable portions include the endoscope, the treatment tool, the overtube, and the like. For the endoscope, the treatment tool, or the overtube, those different in length, balloon position, and the like are prepared.

FIG. 21 shows a modification of an operation device for manually operating an electric endoscope. Although an operation device 300 configured to be grasped by a user is shown in FIG. 9, a console-type operation device 320 may be used as shown in FIG. 21. The operation device 320 includes a monitor 325 for displaying an endoscope image, an operation section 321 operated by a right hand, an operation section 322 operated by a left hand, and one or more foot switches 323. Both the operation sections 321 and 322 are capable of, for example, operation input in the vertical and horizontal directions. For example, the vertical/horizontal bending operation of the endoscope 100 is assigned to the vertical/horizontal operation of the operation section 321, the forward/backward operation and the rolling rotation operation of the endoscope 100 are assigned to the vertical/horizontal operation of the operation section 322, and the vertical operation of the raising base of the treatment tool 400 is assigned to the foot switch. The assignment of functions is not limited to this example.

As described above, the information obtainable in the ERCP procedure is only the endoscope image showing the papillary portion. Because there are individual differences in the form of the papillary portion and luminal tissue, it is difficult to specify the insertion position and the insertion direction of the cannula only based on the endoscope image. In order to more accurately assume the position of the opening and the travelling direction of the biliary duct, it is desirable to perform positioning so that the papillary portion is shown at a predetermined position in the image; however, it is difficult to adjust the position of the distal end of the endoscope because, for example, the operation performed at the base end side of the insertion section is not easily transmitted to the distal end section, or because the distal end section of the endoscope is easily displaced from the papillary portion. Although a robotic catheter system may be known for use in ERCP, the prior art does not disclose or suggest any of the above-mentioned problems or subject matter for solving them.

Therefore, the medical system 10 of the present embodiment includes the endoscope 100 and the control device 600. In the endoscope 100, the endoscopic operation is electrically driven, thereby capturing an endoscope image. The endoscopic operation is at least one of forward and backward movement of the insertion section 110, the bending angle of the bending section 102 of the insertion section 110, and the rolling rotation of the insertion section 110. The control device 600 controls the electrically-driven endoscopic operation. After the first positioning for positioning the insertion section 110 with respect to the papillary portion of the duodenum is performed, the control device 600 controls the electrically-driven endoscopic operation based on the endoscope image, thereby performing the second positioning for positioning the distal end section 130 of the insertion section 110 with respect to the papillary portion.

According to the present embodiment, positioning of the insertion section 110 with respect to papillary portion can be done by the first positioning. Then, by performing the second positioning based on the positional relationship between the insertion section 110 thus positioned and the papillary portion, the endoscopic operation near the distal end can be more flexibly controlled by electrical driving. Further, by automatically controlling the electrical driving of the endoscopic operation or the like based on the endoscope image in the second positioning, the distal end section 130 of the insertion section 110 is automatically positioned with respect to the papillary portion. As a result, it is not necessary to perform minute position adjustment by non-electric manual operation, and it is possible to assist the cannulation procedure performed by an inexperienced operator or the like.

The endoscopic operation is described above with reference to FIG. 8. The papillary portion of the duodenum is described about with reference to FIG. 1. The first positioning and the second positioning are described above with reference to FIG. 6.

In the present embodiment, the medical system 10 may include a holding member that performs the first positioning of the insertion section 110 by holding the insertion section 110.

According to the present embodiment, since the insertion section 110 is held by the holding member in the first positioning, the electrically-driven endoscopic operation is easily transmitted from the base end side to the distal end side of the insertion section 110 in the second positioning; further, the insertion section 110 is held so that the distal end section 130 of the endoscope 100 is prevented from displacing from the papillary portion.

The holding member corresponds to the overtube 710, the balloon 720, or both described with reference to FIG. 5, 8, 16a, 16b, or 17, etc. Alternatively, the holding member may be the balloon 730 described in FIG. 18, the suction cap 740 described in FIG. 19, or the like.

In the present embodiment, the control device 600 may perform the second positioning for slightly adjusting the position of the distal end section 130 of the insertion section 110 by an endoscopic operation on a distal end side relative to the holding member holding the insertion section 110.

According to the present embodiment, in the first positioning, the position of the distal end section 130 with respect to papillary portion is roughly adjusted, and the insertion section 110 is held by the holding member. As a result, the electrically-driven endoscopic operation is accurately performed, and the distal end section 130 is prevented from displacing from the papillary portion. Therefore, in the second positioning, the position of the distal end section 130 of the endoscope 100 can be slightly adjusted with respect to the papillary portion by the endoscopic operation on a distal end side relative to the holding member.

The term “on a distal end side relative to the holding member” means a portion of the holding member closer to the distal end than a portion to be hardened, fixed, etc. to restrict the endoscopic operation. This is described above with reference to FIG. 8.

Further, in the present embodiment, the control device 600 may perform the second positioning for controlling the electrically-driven endoscopic operation in a manner such that the image of the papillary portion is captured at a position registered in advance on the endoscope image.

According to the present embodiment, it is possible to obtain an endoscope image in which the papillary portion is located at the position registered in advance. As a result, the papillary portion appears in a familiar position and can thus be easily compared with those in the past cases or experiences. Therefore, the operator can easily judge the position of the opening of the luminal tissue and the travelling direction of the biliary duct by viewing the endoscope image. Further, since the positioning is performed by electrical driving, it is possible to obtain an endoscope image in which the papillary portion is located at a position registered in advance without requiring the operator to perform a minute operation. In addition, the control device 600 may perform the second positioning for controlling the electrically-driven endoscopic operation in a manner such that the image of the opening of the luminal tissue is captured at a position registered in advance in the endoscope image. The operator can grasp the position of the opening of the luminal tissue by viewing the endoscope image. For example, even in a case where the opening of the luminal tissue is closed in the image of the papillary portion and therefore is difficult to be visually recognized, the operator can presume that the opening of the luminal tissue is present at a predetermined position in the endoscope image.

The position registered in advance is described above, for example, with regard to FIG. 6 or FIG. 10 .

In the present embodiment, the papillary portion may include the opening of the luminal tissue. The opening of the luminal tissue may be an opening of the common duct in which the biliary duct and the pancreatic duct merge or an opening of the biliary duct, in the papillary portion of the duodenum.

In the papillary portion of the duodenum, there are various individual differences in the form of the papillary portion and the structure of the luminal tissue. According to the present embodiment, the positioning of the distal end section 130 of the endoscope 100 with respect to the papillary portion of the duodenum with various individual differences can be done by the first positioning and the second positioning. In addition, since the positioning of the papillary portion is automated, it is possible to assist the cannulation in the ERCP procedure.

The papillary portion of the duodenum, the opening of the luminal tissue, and their relationship are described above.

Further, in the present embodiment, the medical system 10 includes a treatment tool 400. The treatment tool 400 is inserted from the insertion opening 190 of the treatment tool 400 of the endoscope 100, and is raised inside the distal end section 130 to thereby protrude from the side surface of the distal end section 130. The control device 600 performs the second positioning for controlling at least one of the endoscopic operation and the raising angle of the treatment tool 400 in a manner such that the treatment tool 400 faces toward the travelling direction of the biliary duct that is presumed from the endoscope image in which the image of the papillary portion is captured.

According to the present embodiment, at least one of the endoscopic operation and the raising angle of the treatment tool 400 is automatically controlled by the second positioning so that the treatment tool 400 faces toward the travelling direction of the biliary duct. As a result, the assumption of the travelling direction of the biliary duct, which is difficult due to individual differences in the papillary portion or the like, is automated, thereby assisting the ERCP procedure performed by an inexperienced operator or the like.

The raising of the treatment tool 400 is described above with regard to FIGS. 14a and 14b. The control for directing the treatment tool 400 toward the travelling direction of the biliary duct is described, for example, with regard to FIG. 10.

Further, in the present embodiment, the holding member may include a first holding member for holding the insertion section 110 with respect to the organ in which the papillary portion is present.

Specifically, the first holding member may be a member that holds the insertion section 110 with respect to the duodenum in a state in which the distal end section 130 of the endoscope 100 reaches the papillary portion of the duodenum.

Further, the first holding member may be provided closer to the base end of the insertion section 110 than the base end of the bending section 102.

Further, the first holding member may be a balloon 720 that is inflated and comes in contact with an organ, thereby holding the insertion section 110 with respect to the organ.

Since the insertion opening 190 is held in the organ by the first holding member, it becomes possible to perform the first positioning of the insertion section 110 with respect to the papillary portion in the organ, thereby flexibly controlling the endoscopic operation near the distal end by electrical driving in the subsequent second positioning.

The first holding member corresponds to the balloon 720 described above with reference to FIG. 5, 8 or 17, etc. Alternatively, the first holding member may be the balloon 730 described in FIG. 18, the suction cap 740 described in FIG. 19, or the like.

Further, in the present embodiment, the holding member may include a second holding member for holding the route of the insertion section 110 to the first holding member for holding the insertion section with respect to the organ.

The second holding member may be an overtube 710 with a variable hardness that holds the route of the insertion section 110 by being hardened.

Since the insertion opening 190 is held in the organ by the second holding member, it becomes possible to perform the first positioning of the insertion section 110 with respect to the opening of the luminal tissue in the organ, thereby flexibly controlling the endoscopic operation near the distal end section 130 by electrical driving in the subsequent second positioning.

The second holding member corresponds to the overtube 710 described with reference to FIG. 5, 8, 16a, 16b, or 17, etc. The variable hardness of the overtube 710 is described above with reference to FIGS. 16a and 16b.

Further, in the medical system 10, the electrical driving of the bending movement of the endoscope 100 is not limited to the structure of the present embodiment. For example, it may be structured such that an attachment equipped with an electric motor is detachably attached to a bending operation knob of a non-electrically-driven endoscope. The drive control device 200 and the attachment are structured to communicate with each other, and, upon reception of a bending control signal from the drive control device 200, the attachment is driven to perform the bending. In this case, the manual control and the automatic control can be switched by attaching and detaching the attachment. It may also be arranged such that a handle capable of controlling the driving of the drive control device 200 is detachably attached to a motor for bending control corresponding to the drive control device 200. In this case, the manual control and the automatic control can be switched by attaching and detaching the handle.

The present embodiment may be implemented as a cannulation method as follows. More specifically, the cannulation method uses the endoscope 100. In the endoscope 100, the endoscopic operation is electrically driven, thereby capturing an endoscope image. The endoscopic operation is at least one of forward and backward movement of the insertion section 110, the bending angle of the bending section 102 of the insertion section 110, and the rolling rotation of the insertion section 110. The cannulation method includes a step of inserting the insertion section 110 of the endoscope 100 into a body. The cannulation method includes a step of performing the first positioning for positioning the insertion section 110 with respect to the papillary portion of the duodenum. The cannulation method includes, after the step of performing the first positioning, a step of performing the second positioning for positioning the distal end section 130 of the insertion section 110 with respect to the papillary portion by controlling electrically-driven endoscopic operation based on an endoscope image. The cannulation method includes, after the step of performing the second positioning, a step of performing cannulation from the papillary portion to the biliary duct.

In the present embodiment, in the step of performing the first positioning, the first positioning of the insertion section 110 may be performed by a holding member that holds the insertion section 110.

Further, in the present embodiment, in the step of performing the second positioning, the position of the distal end section 130 of the endoscope 100 may be slightly adjusted by an endoscopic operation on a distal end side relative to the holding member holding the insertion section 110.

Further, in the present embodiment, in the step of performing the second positioning, the electrically-driven endoscopic operation may be controlled so that the image of the papillary portion is captured at a position registered in advance in the endoscope image.

Further, in the present embodiment, the treatment tool 400 is inserted from the insertion opening 190 of the treatment tool 400 of the endoscope 100, and is also raised inside the distal end section 130 to thereby protrude from the side surface of the distal end section 130. In the step of performing the second positioning, it is possible to control at least one of the endoscopic operation and the raising angle of the treatment tool 400 in a manner such that the treatment tool 400 faces toward the travelling direction of the biliary duct that is presumed from the endoscope image in which the image of the papillary portion is captured.

The present embodiment may also be performed as a method of operating the medical system 10 as follows. More specifically, the method of operating the medical system 10 uses the endoscope 100. In the endoscope 100, the endoscopic operation is electrically driven, thereby capturing an endoscope image. The endoscopic operation is at least one of forward and backward movement of the insertion section 110, the bending angle of the bending section 102 of the insertion section 110, and the rolling rotation of the insertion section 110. The method of operating the medical system 10 includes a step of performing the first positioning for positioning the insertion section 110 with respect to the papillary portion of the duodenum. The method of operating the medical system 10 includes, after the step of performing the first positioning, a step of performing the second positioning for positioning the distal end section 130 of the insertion section 110 with respect to the papillary portion by controlling electrically-driven endoscopic operation based on an endoscope image. In the method of operating the medical system 10, the subject of each step is the medical system 10.

In accordance with an aspect of the disclosed embodiments, a medical system can be provided. The medical system comprising:

a treatment tool configured to be inserted into an endoscope and including a sheath whose forward and backward movement is electrically driven;

an operation device for operating the electrically-driven forward and backward movement of the sheath; and

a controller comprising hardware,

the operation device comprising:
a base;
an operation sheath disposed to be longitudinally slidable with respect to the base; and
a sliding operation detection sensor configured to detect a sliding operation of the operation sheath,

the controller being configured to control the electrically-driven forward and backward movement of the sheath of the treatment tool based on detected information of the sliding operation detected by the sliding operation detection sensor.

The present embodiment relates to an operation device in an electric medical system. The following describes an example where the medical system is applied to the ERCP procedures; however, the application of the medical system is not limited to ERCP and the medical system can be applied to any procedures including operations of moving a treatment tool of an endoscope forward and backward.

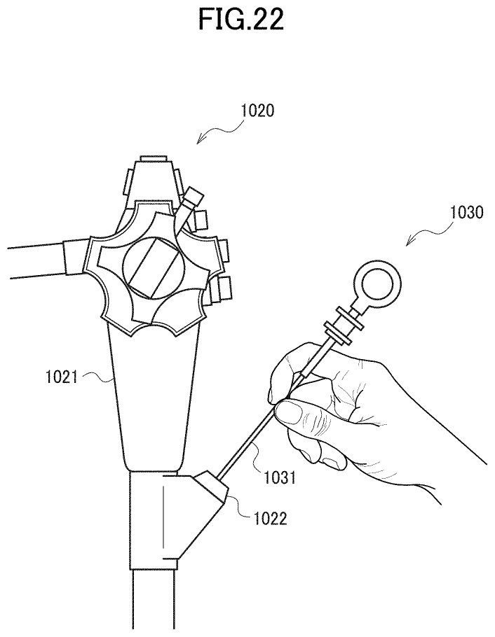

FIG. 22 is a diagram showing an operation of inserting a cannula when using non-electric endoscope and cannula. The operation section 1020 of the endoscope has a grip section 1021 and an insertion opening 1022 provided at the base end of the insertion section. The sheath 1031 of the cannula 1030 is inserted from the insertion opening 1022 into the treatment tool channel, and the distal end of the sheath 1031 protrudes from the distal end section of the endoscope toward the papillary portion. The operator holds the grip section 1021 of the operation section 1020 with one hand to operate the endoscope, and pushes and pulls the sheath 1031 with the other hand to move the sheath 1031 protruding from the distal end section of the endoscope forward and backward.

The operator inserts the cannula into the biliary duct, which is not shown in the endoscope image, while presuming the insertion position and the insertion direction of the cannula from an endoscope image in which the papillary portion is shown as in a to d in FIG. 2. At this time, as explained in FIGS. 2 and 3, it is difficult to appropriately insert the cannula into the biliary duct without, for example, inserting it into the pancreatic duct, because the papillary portion and the luminal tissue have a wide variety of forms. In order to properly perform this difficult operation, the sense of feel when pushing and pulling the non-electric sheath 1031 shown in FIG. 22 by hand is important.

When the cannula is electrically driven, the electric cannula is operated by operating the operation device instead of manually operating the sheath 1031 of the cannula 1030 as shown in FIG. 22. Commonly used operation input sections include buttons, dials, D-pads, levers, and the like. However, these operation input sections are incapable of reproducing the sense of feel in pushing and pulling the non-electric sheath 1031 shown in FIG. 22 by hand. For example, in the first place, these operation input sections are not configured to push and pull the sheath 1031. Also, if it is a non-electric cannula, when the distal end of the cannula hits the narrowed portion or an obstacle, the force feedback is felt in the hand pushing the sheath 1031; however, such a force feedback is not appropriately performed in the operation input sections described above. Even if a kind of force feedback is made, it would not be a natural feedback because it is not an operation of pushing or pulling the sheath 1031.

Medical System According to Another Embodiment

Therefore, the present embodiment provides an operation device capable of reproducing the sense of feel in pushing and pulling the sheath of the treatment tool by hand in an electric medical system, thereby assisting the ERCP procedures. The details of this structure are described below.

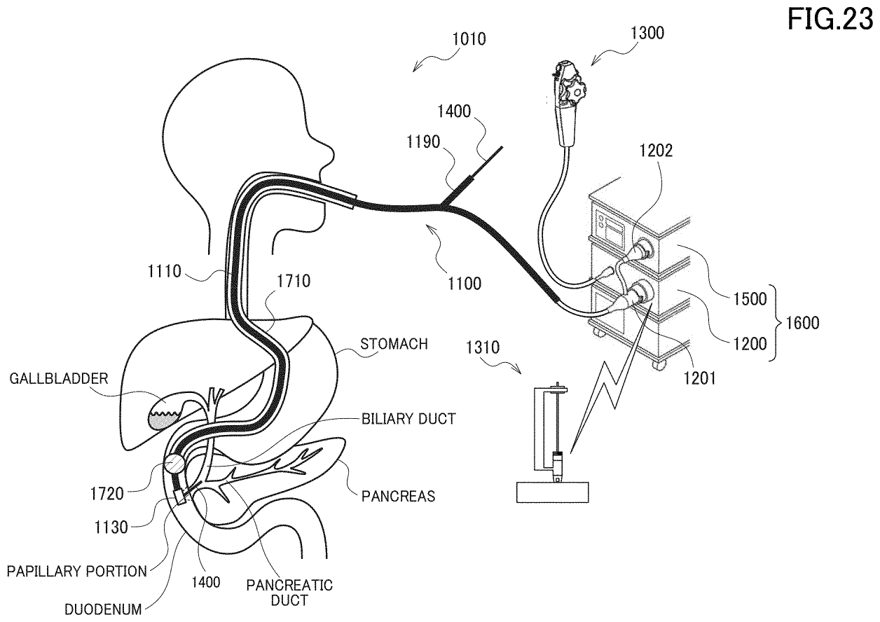

FIG. 23 shows a basic configuration example of a medical system 1010 according to the present embodiment. The medical system 1010 includes an endoscope 1100, an overtube 1710, a balloon 1720, a treatment tool 1400, a control device 1600, and operation devices 1300 and 1301. The following describes an example in which the endoscope and the treatment tool are electrically driven; however, the electric driving of the endoscope is not indispensable. Further, it is sufficient that at least the forward/backward movement of the treatment tool is electrically driven.

The overtube 1710 is a tube with a variable hardness that covers the insertion section 1110 of the endoscope 1100. The balloon 1720 is provided near the distal end on the outer side of the overtube 1710. The operator inserts the endoscope 1100 and the overtube 1710, which is in a soft state, to the duodenum, inflates the balloon 1720 to fix a portion around the distal end of the overtube 1710 to the duodenum, and hardens the overtube 1710. When the endoscope 1100 and the overtube 1710 are inserted into the body, at least the bending section of the insertion section 1110 is exposed from the distal end of the overtube 1710. The bending section refers to a section structured to be bent at an angle corresponding to the bending operation in the vicinity of the distal end of the insertion section 1110. The base end of the overtube 1710 is present outside the body. The base end side of the insertion section 1110 is exposed from the base end of the overtube 1710. Although the example herein uses the overtube 1710 and the balloon 1720, they may be omitted.

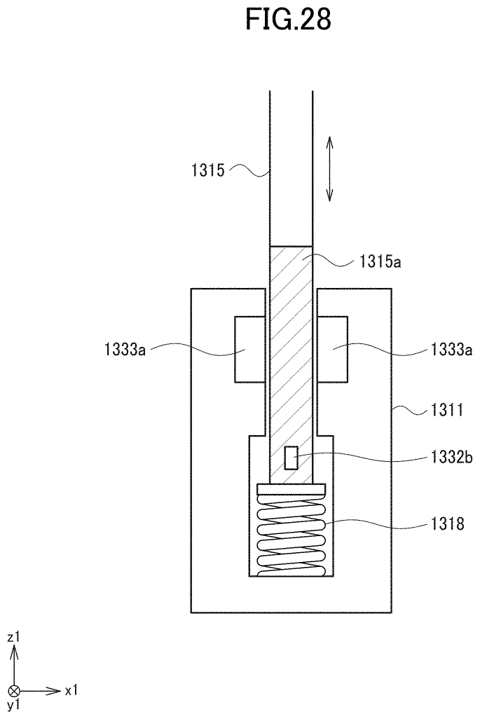

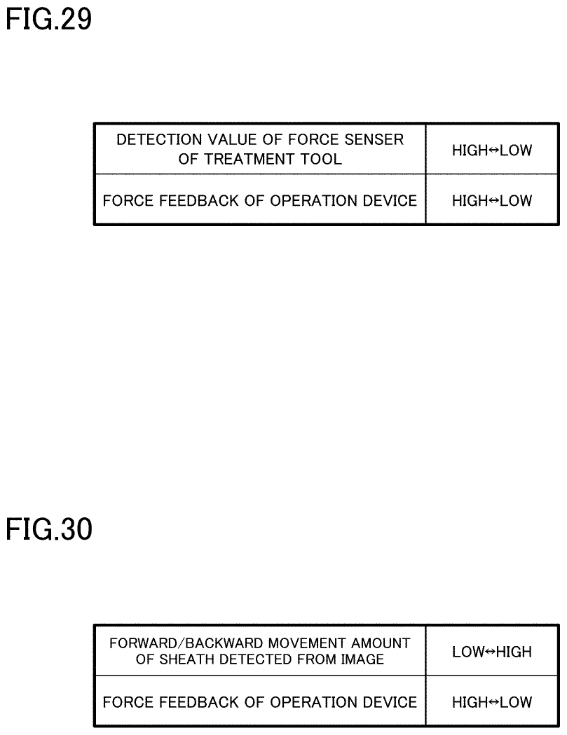

An insertion opening 1190 of the treatment tool is provided at the base end side of the insertion section 1110, and a treatment tool channel for allowing the treatment tool 1400 to pass through from the insertion opening 1190 to the opening of the distal end section 1130 is provided inside the insertion section 1110. The insertion opening 1190 of the treatment tool is also called a forceps opening; however, the treatment tool to be used is not limited to forceps. The operations of the treatment tool 1400, which are forward and backward movement, bending of the distal end section, and rolling rotation, are electrically driven. The operation device 1310 is an operation device for operating the treatment tool 1400, and is hereinafter also referred to as a treatment tool operation device. The operation device 1310 is connected to the drive control device 1200 via, for example, wireless communication. The drive control device 1200 receives an operation input signal from the operation device 1310 and controls the operation of the treatment tool 1400 based on the operational input signal. The operation device 1310 and the drive control device 1200 may be connected via wired connection using cables, connectors, etc. The detailed configuration example of the treatment tool 1400 is described later with reference to FIG. 28, and the detailed configuration example of the operation device 1310 is described later with reference to FIG. 29 onward.

The endoscope 1100 is detachably connected to a control device 1600 using connectors 1201 and 1202. The control device 1600 includes a drive control device 1200 to which the connector 1201 is connected, and a video control device 1500 to which the connector 1202 is connected. The drive control device 1200 controls the electrical driving of the endoscope 1100 via the connector 1201. The drive control device 1200 is connected to the operation device 1300 for enabling manual operation of the electrical driving of the endoscope 1100. Hereinafter, the operation device 1300 is also referred to as an endoscope operation device. The video control device 1500 receives an image signal from a camera provided at the distal end section 1130 of the endoscope 1100 via the connector 1202, generates a display image from the image signal, and displays it on a display device (not shown). In FIG. 6, the drive control device 1200 and the video control device 1500 are shown as separate devices, but they may be structured as a single device. In this case, the connectors 1201 and 1202 may be integrated into a single connector.

The term “electrical driving” means that the treatment tool 1400 is driven by a motor or the like based on an electrical signal so as to control the treatment tool motion. The electrically-driven manual operation means that the operation device 1310 converts the operation input made by the operator from the operation device 1310 into an electrical signal, thereby driving the treatment tool 1400 by a motor or the like based on the electrical signal. The meaning of electrical driving and the meaning of electrically-driven manual operation are the same also for the endoscopic operation.

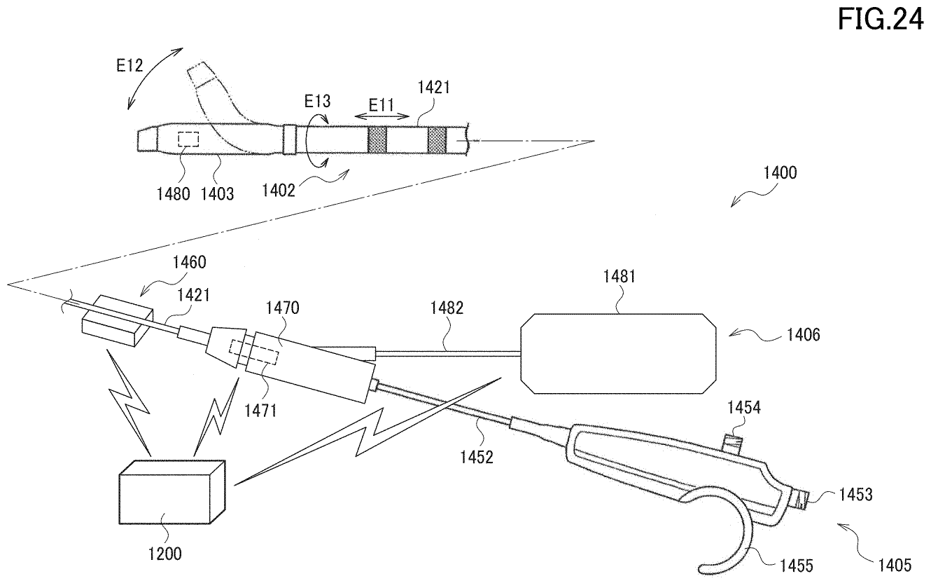

FIG. 24 shows a detailed configuration example of an electric treatment tool 1400. Herein, as an example of the treatment tool 1400, a cannula in which the forward and backward movement, the bending movement, and the rolling rotation are electrically driven is shown. However, it is sufficient that the forward and backward movement is electrically driven, and at least one of the bending movement and the rolling rotation may be non-electrically driven. The bending mechanism in the distal end section is not indispensable and may be omitted regardless of whether or not it is electrically driven or non-electrically driven.

The treatment tool 1400 includes a long-length insertion section 1402 extending in the axial direction, a bending section 1403 capable of bending movement, a bending driving section 1406 for electrically driving the bending section 1403, and an operation section 1405 for inserting a contrast agent or a guide wire.

In FIG. 24, the distal end side of the tube 1421 is enlarged. The insertion section 1402 has a tube 1421, and the bending section 1403 is connected to the distal end of the tube 1421. In the example in FIG. 24, the tube 1421 corresponds to the sheath of the treatment tool. The tube 1421 may hereinafter also be referred to as a sheath. A force senser 1480 is provided in the vicinity of the distal end of the bending section 1403. The force senser 1480 can be provided in areas not involved in the shape change by the bending movement. FIG. 24 shows an example in which a single force senser is provided, but it is also possible to provide a plurality of force sensers. Note that the force senser 1480 may be omitted depending on the method of the force feedback, or if the force feedback is not performed.

An example of the force senser 1480 is a strain gauge. A strain gage is a mechanical sensor that measures the strain of an object on which the strain gage is provided. The distal end of the bending movement section 1403 is deformed as it comes in contact with organs, and the like, and the strain gauge detects the deformation, thereby detecting the stress of the contact. The strain gage has a thin insulator and a metal foil resistor provided on the insulator. When stress is applied to an object to which the strain gage is attached, the strain gage is deformed together with the object, and the resistance of the metal foil resistor is changed by the deformation. The drive control device 1200 measures this resistance, thereby detecting the stress.

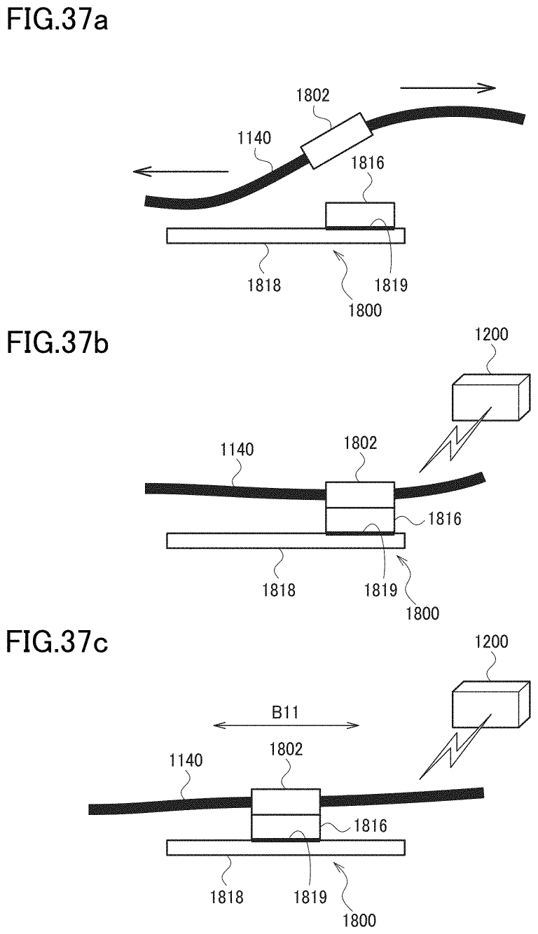

The tube 1421 of the insertion section 1402 is detachable from the forward/backward drive device 1460, and has a structure similar to, for example, the forward/backward drive device 1800 for use in endoscopes, which is described later with reference to FIG. 37. That is, the tube 1421 has an attachment detachable from the forward/backward drive device 1460, and the attachment is attached to the motor of the forward/backward drive device 1460, thereby enabling the forward/backward movement, i.e., in the direction of arrow E11 to be electrically driven. The drive control device 1200 transmits a forward or backward control signal to the motor by wireless communication, and the motor and the attachment move linearly in the axial direction of the tube 1421 based on the control signal, thereby moving the insertion section 1402 forward or backward. The drive control device 1200 and the forward/backward drive device 1460 may be connected by wired connection.

A connector 1470 is connected to the base end of the tube 1421. The bending driving section 1406 and the operation section 1405 are connected to the connector 1470.

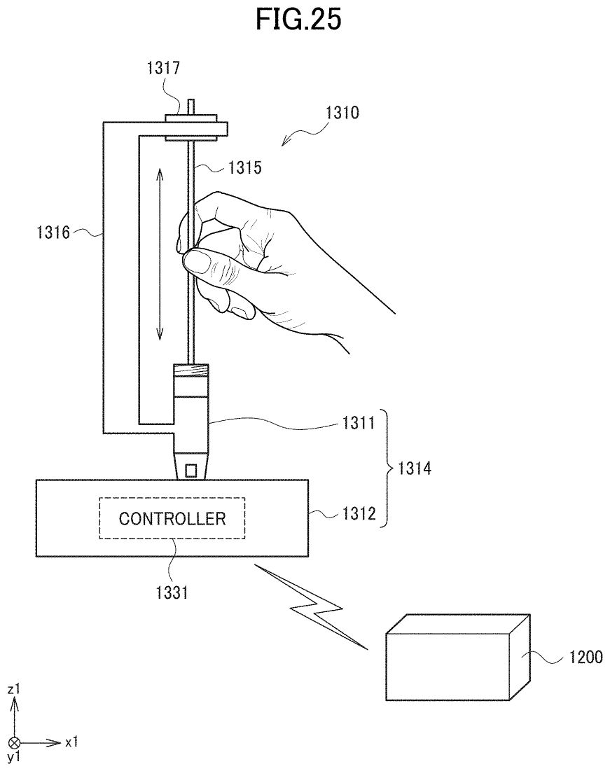

The bending driving section 1406 includes a connecting tube 1482, one end of which is connected to the connector 1470, and a motor 1481 connected to the other end of the connecting tube 1482. Inside the tube 1421, the connector 1470, and the connecting tube 1482, a wire for connecting the bending section 1403 and the motor 1481 is provided. The drive control device 1200 transmits a bending movement control signal to the motor 1481 by wireless communication, and the motor 1481 drives the wire based on the control signal, thereby bending the bending section 1403. For example, the bending movement of the bending section 1403 can be realized by a structure with a plurality of bending pieces, similarly in the bending section of the endoscope. The treatment tool 1400 in FIG. 25 is capable of bending in one direction, i.e., the direction of arrow E12. Note that the drive control device 1200 and the motor 1481 may be connected by wired connection.

The operation section 1405 includes a connecting tube 1452, one end of which is connected to the connector 1422, an operation main body 1451 connected to the other end of the connecting tube 1452, a first opening 1453 opened in the axial direction of the connecting tube 1452 on the base end side of the operation main body 1451, a second opening 1454 opened to the outer surface of the operation main body 1451, and a hook 1455 provided on the operation main body 1451. The hook 1455 has elasticity and is formed in a substantially C-shape, and is used for locking the treatment tool 1400 to the endoscope 1100 or the like. The first opening 1453 and the second opening 1454 are connected to the tube 1421 via the operation main body 1451, the connecting tube 1452, and the connector 1422. By inserting a contrast agent or a guide wire from the first opening 1453 or the second opening 1454, the contrast agent can be injected into the body or the guide wire can be inserted into the body from the distal end of the treatment tool 1400.

Inside the connector 1470, a motor 1471 is provided to electrically drive the rolling rotation of the insertion section 1402. The connector main body of the connector 1470 has a cylindrical shape, and a cylindrical member coaxial with the cylinder is rotatably provided inside the connector main body. The base end section of the tube 1421 of the insertion section 1402 is fixed to the outside of the cylindrical member. As a result, the tube 1421 and the cylindrical member are rotatable with respect to the connector main body about the axial direction of the tube 1421. The drive control device 1200 transmits a rolling rotation control signal to the motor 1471 by wireless communication, and the motor 1471 rotates the base end section of the tube 1421 with respect to the connector main body based on the control signal, thereby allowing the insertion section 1402 to undergo rolling rotation, i.e., the direction of arrow E13. Note that the drive control device 1200 and the motor 1471 may be connected by wired connection.

Detailed Configuration Example of Treatment Tool Operation Device

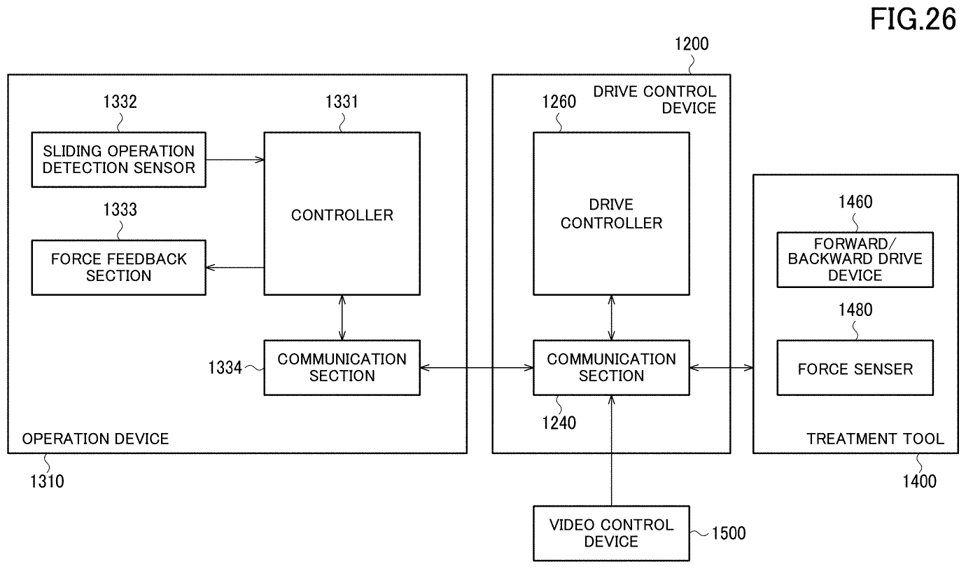

FIG. 25 shows a first detailed configuration example of a treatment tool operation device. The operation device 1310 includes a base 1314, an operation sheath 1315, an arm section 1316, and a sheath holding section 1317. In FIG. 26, the axial direction of the operation sheath 1315 is referred to as the z1 direction, and the two directions that are orthogonal to the z1 direction and also orthogonal to each other are referred to as the x1 direction and the y1 direction, respectively.

The operation sheath 1315 is a sheath for use in operation and is provided separately from the sheath of the treatment tool 1400. The operation sheath 1315 is a replica of the sheath of the treatment tool 1400. However, the operation sheath 1315 may not be exactly the same as the sheath of the treatment tool 1400. For example, the material, the diameter, the hardness, etc. of the operation sheath 1315 may be different from the material, the diameter, the hardness, etc. of the sheath of the treatment tool 1400. The operation sheath 1315 does not necessarily have to be tubular, but can be a long cylindrical shape, or the like.

The base 1314 includes a first base 1311 that holds one end of the operation sheath 1315 and a second base 1312 that stores a controller 1331 for controlling the operation device 1310. The second base 1312 may be omitted; in this case, the controller 1331 may be stored in the first base 1311, or the like.

The operation sheath 1315 is slidable in the axial direction with respect to the first base 1311. For example, the −z1 direction corresponds to the forward direction of the sheath of the treatment tool 1400. In this case, when the operator slides the operation sheath 1315 in the −z1 direction, the sheath of the treatment tool 1400 moves forward, and when the operator slides the operation sheath 1315 in the +z1 direction, the sheath of the treatment tool 1400 moves backward. The sliding of the operation sheath 1315 and the forward/backward movement of the sheath of the treatment tool 1400 may be associated with each other in various ways. As an example, the amount of the forward/backward movement of the sheath of the treatment tool 1400 may be proportional to the sliding amount of the operation sheath 1315. Alternatively, the treatment tool 1400 may continue to move forward when the operation sheath 1315 is pushed in by being slid in the −z1 direction and kept in that state. The same can be said for the backward movement.

The arm section 1316 is a rigid member for connecting the first base 1311 and the sheath holding section 1317 and supports the sheath holding section 1317 on the other end of the operation sheath 1315. The arm section 1316 includes a first portion that is parallel to the operation sheath 1315, a second portion extending in the x1-direction from one end of the first portion and connected to first base 1311, and a third portion extending in the x1-direction from the other end of the first portion and supporting the sheath holding section 1317.

The sheath holding section 1317 is a member that holds the other end of the operation sheath 1315 so that the operation sheath 1315 is slidable in the axial direction. The sheath holding section 1317 is provided with a hole that penetrates through it in the z1 direction. The operation sheath 1315 is inserted in the hole, and the operation sheath 1315 is slidable in the axial direction along the hole.

If the treatment tool 1400 has the bending driving section 1406 and the motor 1471 for the rolling rotation as shown in FIG. 24, the operation device 1310 may further include a rolling rotation operation section and a bending operation section by way of buttons, dials, levers, etc. Alternatively, the bending operation section and the rolling rotation operation section for the treatment tool may be provided in the operation device 1300 for endoscopes.

According to the present embodiment described above, the operator can operate the forward and backward movement of the electric treatment tool by pushing and pulling the operation sheath 1315, which is a replica of the sheath of the treatment tool, in the axial direction. As a result, the sense of feel in the forward and backward movement of the non-electric treatment tool can be reproduced in the operation device of an electric treatment tool, thereby obtaining a natural sense of feel in operation in the procedure, such as cannulation. In addition, by performing the force feedback as described below, the hand sensation in operating the non-electric treatment tool can be reproduced in the operation device of an electric treatment tool. Also, by performing the force feedback with respect to the operation sheath 1315, which is a replica of the sheath of the treatment tool, a natural force feedback similar to when operating a non-electric treatment tool can be obtained.

FIG. 26 shows a block configuration example of the medical system 1010. The following describes a block configuration example of a portion of the treatment tool involved in the forward/backward movement operations. The medical system 1010 includes an operation device 1310, a treatment tool 1400, a drive control device 1200, and a video control device 1500. The operation device 1310 includes a controller 1331, a sliding operation detection sensor 1332, a force feedback section 1333, and a communication section 1334. The drive control device 1200 includes a drive controller 1260 and a communication section 1240 (e.g., transceiver). The treatment tool 1400 includes a forward/backward drive device 1460 and a force senser 1480.

The sliding operation detection sensor 1332 and the force feedback section 1333 are stored in, for example, the first base 1311, and the controller 1331 and the communication section 1334 are stored in, for example, the second base 1312. However, there is no such limitation, and some or all of the controller 1331, the sliding operation detection sensor 1332, the force feedback section 1333 and the communication section 1334 may be stored in a section other than the base 1314, for example, in the arm section 1316 or the like.

First, the operation of the medical system 1010 when the treatment tool 1400 moved forward or backward in response to the sliding operation is described below.

The sliding operation detection sensor 1332 detects the sliding operation of the operation sheath 1315. Specifically, the sliding operation detection sensor 1332 detects the sliding direction and the sliding amount of the operation sheath 1315 in the axial direction. The sliding operation detection sensor 1332 is a force senser that detects the stress applied to the operation sheath 1315 by the sliding operation, or an optical sensor or the like that detects the sliding movement itself of the operation sheath 1315.

The controller 1331 obtains information regarding the sliding direction and the sliding amount based on the detection signal of the sliding operation detection sensor 1332, and transmits the information from the communication section 1334 to the communication section 1240 of the drive control device 1200. The communication section 1334 of the operation device 1310 and the communication section 1240 of the drive control device 1200 are connected, for example, by wireless communication; however, they may also be connected by wired communication.

The drive controller 1260 generates a control signal for controlling the forward/backward movement direction and the forward/backward movement amount of the treatment tool 1400 based on the received information of the sliding direction and the sliding amount. The drive controller 1260 transmits the control signal to the forward/backward drive device 1460 of the treatment tool 1400 via the communication section 1240. The forward/backward drive device 1460 causes the sheath of the treatment tool 1400 to move forward or backward so that the treatment tool 1400 moves forward/backward in the forward/backward movement direction or with the forward/backward movement amount.

Next, the operation of the medical system 1010 in performing the force feedback to the operation device 1310 is described below.

The force senser 1480 detects the stress applied to the distal end section of the treatment tool 1400 from organs or tissues. The drive controller 1260 receives a detection signal from the force senser 1480 via the communication section 1240. The drive controller 1260 generates a control signal for the force feedback based on the detection signal of the force senser 1480, and transmits the control signal to the communication section 1334 of the operation device 1310 via the communication section 1240. The control signal for the force feedback is a signal indicating the intensity of the feedback, i.e., the intensity of the reaction force felt in the hand when sliding the operation sheath 1315.

The controller 1331 of the operation device 1310 controls the force feedback section 1333 based on the control signal for the force feedback. The force feedback section 1333 generates a force in the direction opposite to the sliding direction of the operation sheath 1315, thereby generating a reaction force against the sliding operation. The force feedback section 1333 is, for example, an electromagnetic coil, which is described later. However, the force feedback section 1333 may include a roller in contact with the operation sheath 1315 and a motor that rotates the roller, or may be a mechanical brake that provides resistance to the sliding operation by frictional force. The resistance due to the frictional force serves as the reaction force against the sliding operation.

By performing such a force feedback as described above, the operator can perform an insertion operation of the treatment tool while feeling the stress applied to the treatment tool, for example, when the treatment tool comes in contact with a tissue.

The controller 1331 is, for example, a processor such as a CPU (Central Processing Unit), a GPU (Graphics Processing Unit), a DSP (Digital Signal Processor), or the like. For example, the operation device 1310 includes a storage section (not shown), which stores a computer-readable program in which the functions of the controller 1331 are described. The functions of the controller 1331 are implemented as processes as the processor executes the program. The drive controller 1260 is, for example, a processor such as a CPU (Central Processing Unit), a GPU (Graphics Processing Unit), a DSP (Digital Signal Processor), or the like. For example, the drive control device 1200 includes a storage section (not shown), which stores a computer-readable program in which the functions of the drive controller 1260 are described. The functions of the drive controller 1260 are implemented as processes as the processor executes the program.

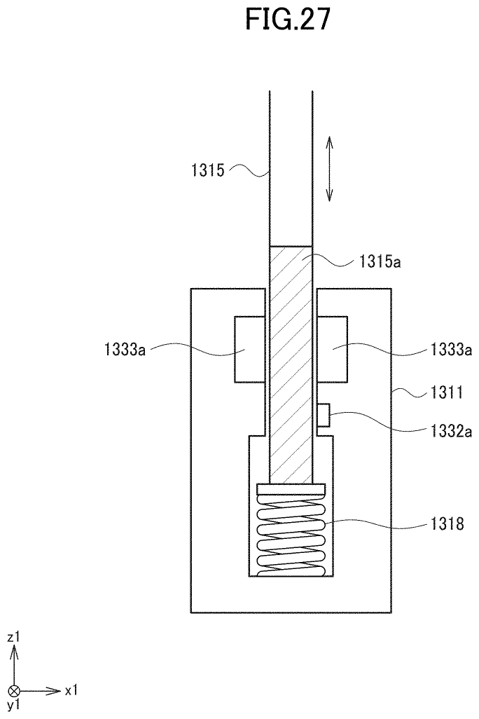

FIG. 27 shows a first detailed configuration example of the first base 1311. The first base 1311 is provided with a spring 1318, an electromagnetic coil 1333a, and an optical sensor 1332a.

The first base 1311 has a hole through which one end of the operation sheath 1315 penetrates in the z1 direction. The bottom of the hole in the first base 1311 and one end of the operation sheath 1315 are connected by the spring 1318, which extends and contracts in the z1 direction. This hole and the spring 1318 enable the first base 1311 to hold the operation sheath 1315 while allowing it to be slidable.

One end of the operation sheath 1315 is made of a metal member 1315a. The parts of the operation sheath 1315 other than the metal member 1315a, i.e., the parts to be slid by hand, is made of a resin, for example. The electromagnetic coil 1333a is provided around the hole through which the metal member 1315a of the operation sheath 1315 passes, and the axis of the electromagnetic coil 1333a is parallel to the penetration direction of the hole. The electromagnetic coil 1333a corresponds to the force feedback section 1333. The controller 1331 controls the current flowing in the electromagnetic coil 1333a, and the electromagnetic coil 1333a exerts a force in the z1 direction on the metal member 1315a according to the current. This controls the force feedback.

The optical sensor 1332a is provided around the hole through which the metal member 1315a of the operation sheath 1315 passes, so that the light emission is directed toward the operation sheath 1315. The optical sensor 1332a corresponds to the sliding operation detection sensor 1332. The optical sensor 1332a detects the sliding direction and the sliding amount of the operation sheath 1315 by sensing the reflected light from the operation sheath 1315.

FIG. 28 shows a second detailed configuration example of the first base 1311. In FIG. 28, a strain gage 1332b is provided as the sliding operation detection sensor 1332. The principle of the strain gage is as described in FIG. 24. The strain gage 1332b is disposed on one end of the operation sheath 1315 and is adhered, for example, to the surface of the metal member 1315a. When the operation sheath 1315 is slid, the spring 1318 expands and contracts, and the metal member 1315a receives a stress from the spring 1318. The strain gage 1332b detects the sliding direction and the sliding amount by detecting the stress. FIG. 28 shows an example in which a single strain gage 1332b is provided; however, a plurality of strain gages may be provided on one end of the operation sheath 1315.

FIG. 29 shows an example of control of force feedback. As described above, the drive controller 1260 controls the force feedback based on the detection signal of the force senser 1480 provided in the treatment tool 1400. At this time, the drive controller 1260 increases the force feedback as the detection value of the force increases. That is, as the force exerted to the distal end of the treatment tool 1400 increases, the drive controller 1260 increases the reaction force against the sliding operation.

The means for detecting the reaction force exerted to the treatment tool 1400 from an organ or tissue is not limited to the force senser. For example, it is possible to detect the movement of the sheath of the treatment tool 1400, and allow the drive controller 1260 to control the force feedback based on the movement. For example, in FIG. 26, the communication section 1240 of the drive control device 1200 receives an endoscope image from the video control device 1500. It is assumed that a papillary portion and the sheath of the treatment tool 1400 are shown in this endoscope image. It is sufficient that only a part of the sheath of the treatment tool 1400 is shown in the endoscope image; the distal end of the treatment tool 1400 may be inserted in the biliary duct. The drive controller 1260 detects the forward/backward movement amount in the sheath's axial direction from the endoscope image, and controls the force feedback based on the forward/backward movement amount.

FIG. 30 shows an example of control of force feedback using an image. The drive controller 1260 increases the force feedback as the forward/backward movement amount detected from the image decreases. More specifically, the drive controller 1260 determines that the reaction force exerted to the distal end of the treatment tool 1400 due to narrowing of the biliary duct or the like is large when the forward/backward movement amount detected from the image is small even though the operation sheath 1315 is operated to slide in the forward direction, and increases the reaction force against the sliding operation. If the force feedback is controlled using an endoscope image, the force senser 1480 may not be provided in the treatment tool 1400.

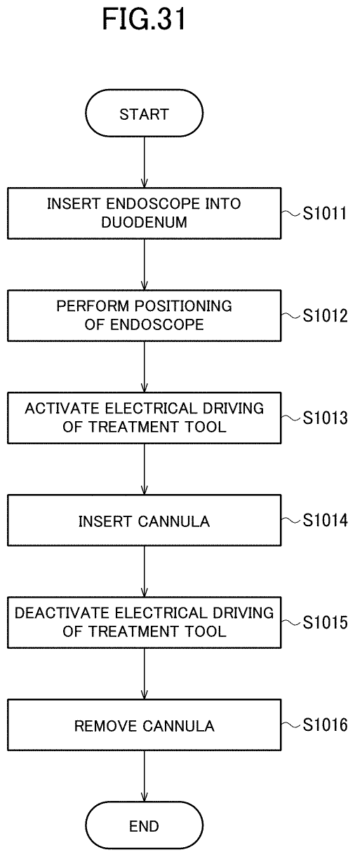

FIG. 31 shows a flow of ERCP procedure using the medical system of the present embodiment. In step S1110, the operator inserts an endoscope into the duodenum. In step S1120, the operator performs positioning of the distal end section of the endoscope with respect to the papillary portion, such as discussed above with regard to FIGS. 1-21. Specifically, the operator performs the positioning of the endoscope so that the camera of the endoscope is directly facing the papillary portion and the papillary portion is shown in the center of the field of view.

In step S1130, the operator activates the electrical driving of the treatment tool. Specifically, the operator inserts the treatment tool through the forceps opening of the endoscope to the distal end section, and after confirming that the distal end of the treatment tool has reached the distal end section of the endoscope, the operator activates the electrical driving of the treatment tool. Alternatively, the operator may insert the treatment tool through the forceps opening of the endoscope to the distal end section after activating the electrical driving of the treatment tool.

When the forward/backward drive device 1460 shown in FIG. 24 is used, the electrical driving of the forward/backward movement is activated by attaching the attachment provided on the tube 1421 to the motor of the forward/backward drive device 1460. Alternatively, the motor may be structured to be detachable by a clutch mechanism or the like, and the non-electrical driving may be switched to the electrical driving by the clutch mechanism. In this case, a button or switch, etc. may be provided in the operation device 1300 or the operation device 1310 to switch between the electrical driving and the non-electrical driving of the treatment tool, and, when the operator operates the button or switch, etc., the electrical driving and the non-electrical driving may be switched by the clutch mechanism.

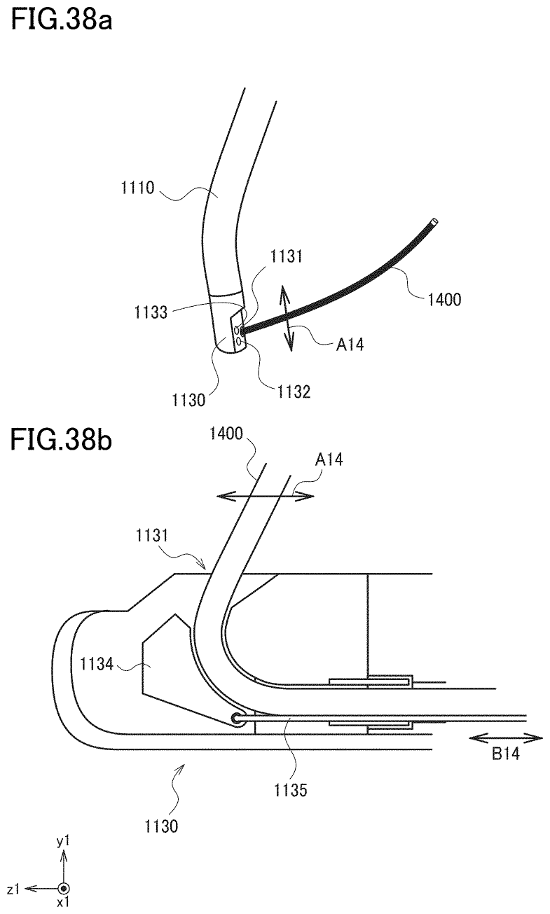

In step S1140, the operator inserts a cannula from the papillary portion to the biliary duct by operating the operation device 1310 for the treatment tool and the operation device 1310 for the endoscope. Specifically, the operator adjusts the rolling rotation of the endoscope and the raising angle of the treatment tool so as to direct the cannula toward the travelling direction of the biliary duct and advance the cannula in the travelling direction, thereby inserting it into the biliary duct. The adjustment of the raising angle is described later with reference to FIG. 38, etc.

The operator injects a contrast agent into the cannula, captures a CT image of the biliary duct or the like, and inserts the guide wire from the cannula into the biliary duct. In step S1150, the operator deactivates the electrical driving of the treatment tool. The method of the deactivation is as described in step S1130. In step S1160, the operator non-electrically removes the cannula from the biliary duct and the endoscope. It may also be arranged such that step S1160 is performed by electrical driving and the electrical driving of the treatment tool may be deactivated thereafter.

In the procedure described above, the endoscope may be operated either by electrical driving or non-electrical driving. The endoscope may also be switched between the electrical driving and the non-electrical driving at any timing during the procedure flow.

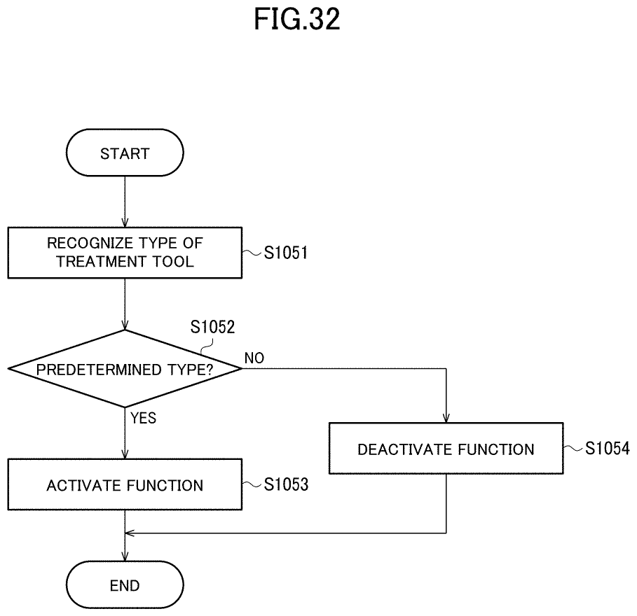

FIG. 32 shows a flow in the case of enabling/disabling functions based on the type of the treatment tool. The functions may be any function related to the treatment tool, e.g., the force feedback of the operation device 1310.

In step S1510, the drive controller 1260 recognizes the type of the treatment tool inserted into the endoscope. Examples of the type of treatment tool include cannula, electronic knife, guide wire, basket, stent and the like. The type of the treatment tool may also be lineup, variation or grades of the same type of treatment tool. For example, it may be arranged such that the treatment tool is provided with a memory storing IDs, and the drive controller 1260 recognizes the type of the treatment tool by retrieving the ID from the memory.

In step S1520, the drive controller 1260 determines whether or not the treatment tool is of the predetermined type. If the type of the treatment tool is the predetermined type, in step S1530, the drive controller 1260 enables the function. If the type of the treatment tool is not a predetermined type, the drive controller 1260 disables the function in step S1540.

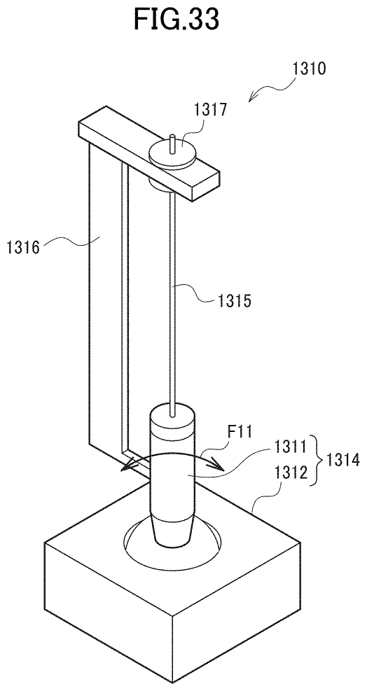

FIG. 33 shows a second detailed configuration example of the treatment tool operation device. In FIG. 33, the operation device 1310 has an operation input section for adjusting the raising angle.

Specifically, the first base 1311 corresponds to the stick part of the joystick. As shown by arrow F11, the first base 1311 and the second base 1312 are structured so that the first base 1311 can be tilted with respect to the second base 1312 in the direction crossing the axial direction of the operation sheath 1315. As described later with reference to FIG. 42, the raising base that raises the treatment tool is electrically driven, and the drive control device 1200 controls the raising angle of the treatment tool in response to the above operation.

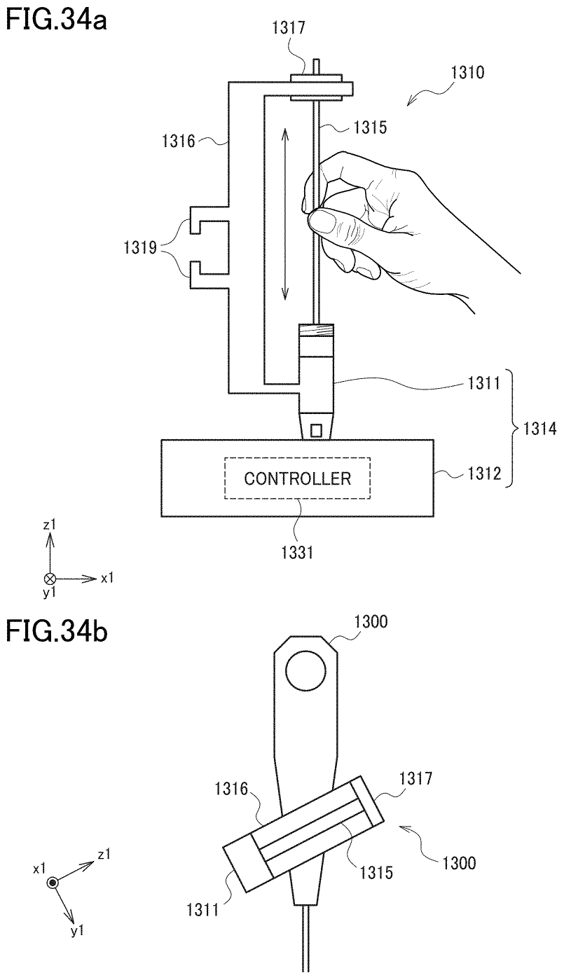

FIGS. 34a and 34b show a third detailed configuration example of the treatment tool operation device. In FIGS. 34a and 34b, the operation device 1310 includes an attachment 1319 for detachably attaching the operation device 1310 to the operation device 1300 for the endoscope.

As shown in FIG. 34a, the attachment 1319 is disposed on the side opposite to the side having the operation sheath 1315 in the arm section 1316. The location of the attachment 1319 is not limited to this and can be anywhere other than the operation sheath 1315. As shown in FIG. 34b, by inserting the operation device 1300 for the endoscope into the attachment 1319, the operation device 1310 for the treatment tool can be attached to the operation device 1300 for the endoscope. The operator holds the operation device 1300 for the endoscope with one hand and operates the operation device 1310 for the treatment tool with the other hand.

It may also be arranged such that the attachment 1319 and the operation device 1300 have connection terminals, and the connection terminals are connected to each other when the operation device 1300 is inserted into the attachment 1319. Then, the operation device 1310 and the drive control device 1200 may establish communication connection with each other via the connection terminals, the operation device 1300, and the cable connecting the operation device 1300 and the drive control device 1200.

Detailed Configuration Example of Medical System

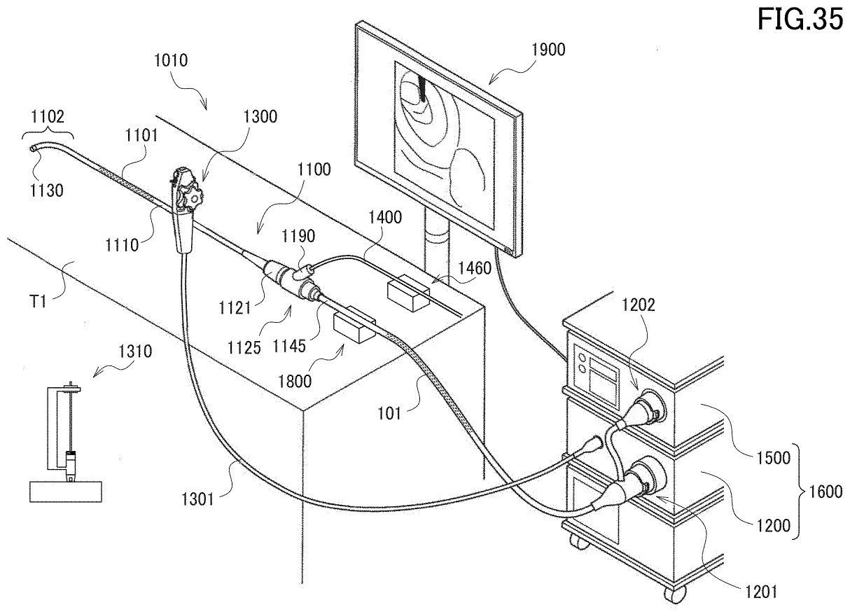

FIG. 35 shows a detailed configuration example of the medical system 1010. Although an example in which the endoscope and the treatment tool are electrically driven is described herein, the endoscope may not be electrically driven. Further, it is sufficient that at least the forward/backward movement of the treatment tool is electrically driven.

The medical system 1010 is a system for observing or treating the inside of the body of a patient lying on an operating table T1. The medical system 1010 includes an endoscope 1100, a control device 1600, operation devices 1300 and 1310, a treatment tool 1400, forward/backward drive devices 1460 and 1800, and a display device 1900. The control device 1600 includes a drive control device 1200 and a video control device 1500.

The endoscope 1100 is a device to be inserted into a lumen of a patient for the observation of an affected part. The side to be inserted into a lumen of a patient is referred to as “distal end side” and the side to be attached to the control device 1600 is referred to as “base end side”. The endoscope 1100 includes an insertion section 1110, a connecting section 1125, an extracorporeal soft section 1145, and connectors 1201 and 1202. The insertion section 1110, the connecting section 1125, the extracorporeal soft section 1145, and the connectors 1201 and 1202 are connected one another in this order from the distal end side.

The insertion section 1110 is a portion to be inserted into a lumen of a patient, and is configured in a soft elongated shape. The insertion section 1110 includes a bending section 1102, an extracorporeal soft section for connecting the base end of the bending section 1102 and the connecting section 1125, and a distal end section 1130 provided at the distal end of the bending section 1102. An internal route 1101 is provided inside the insertion section 1110, the connecting section 1125, and the extracorporeal soft section 1145, and a bending wire passing through the internal route 1101 is connected to the bending section 1102. When the drive control device 1200 drives the wire via the connector 1201, the bending section 1102 bends. Further, a raising base wire connected to the raising base provided at the distal end section 1130 is connected to the connector 1201 through the internal route 1101. As the drive control device 1200 drives the raising base wire, the raising angle of the treatment tool 1400 protruding from the side surface of the distal end section 1130 is changed. The side surface of the distal end section 1130 is provided with a camera, an illumination lens, and an opening of a treatment tool channel. An image signal line for connecting the camera and the connector 1202 is provided in the internal route 1101, and an image signal is transmitted from the camera to the video control device 1500 via the image signal line. The video control device 1500 displays an endoscope image generated from the image signal on the display device 1900.

The connecting section 1125 is provided with an insertion opening 1190 of the treatment tool and a rolling operation section 1121. The treatment tool channel is provided in the internal route 1101, one end of which is open to the distal end section 1130 and the other end of which is open to the insertion opening 1190 of the treatment tool. The treatment tool 1400 is inserted from the insertion opening 1190 of the connecting section 1125 and protrudes into an opening of the distal end section 1130 via the treatment tool channel. The rolling operation section 1121 is attached to the connecting section 1125 so as to be rotatable about the axial direction of the insertion section 1110. By rotating the rolling operation section 1121, the insertion section 1110 undergoes rolling rotation. Further, the rolling rotation can be electrically driven by providing a motor that rotates the rolling operation section 1121 in the connecting section 1125.

The forward/backward drive device 1800 is a drive device for moving the insertion section 1110 of the endoscope 1100 forward and backward by electrical driving. An extracorporeal soft section 1140 is detachable from the forward/backward drive device 1800, and an insertion section 1110 moves forward and backward when the forward/backward drive device 1800 causes the extracorporeal soft section 1140 to slide in the axial direction in a state in which the extracorporeal soft section 1140 is mounted on the forward/backward drive device 1800.

The forward/backward drive device 1460 is a drive device for moving the sheath of the treatment tool 1400 forward and backward by electrical driving. The sheath is detachable from the forward/backward drive device 1460, and, when the forward/backward drive device 1460 causes the sheath to slide in the axial direction in a state in which the sheath is mounted on the forward/backward drive device 1460, the sheath moves forward and backward. Further, as described in FIG. 24, the bending or the rolling rotation may also be electrically driven.

The operation device 1300 of an endoscope is detachably connected to the drive control device 1200 via an operation cable 1301. The operation device 1300 may communicate with the drive control device 1200 through wireless communication instead of wired communication. When an operator operates the operation device 1300, a signal of the operation input is transmitted to the drive control device 1200 via the operation cable 1301, and the drive control device 1200 electrically drives the endoscope 1100 to enable an endoscopic operation corresponding to the operation input based on the signal of the operation input. The operation device 1300 has an operation input section having five or more channels corresponding to the forward and backward movement of the endoscope 1100, the bending movements in two directions and the rolling rotation, and the operation of the raising base. If one or more of these operations are not electrically driven, the operation input section may be omitted. Each operation input section includes, for example, a dial, a joystick, a D-pad, a button, a switch, a touch panel, and the like.

The operation device 1310 for the treatment tool is as described in FIG. 25 to FIG. 34.

The drive control device 1200 electrically drives the endoscope 1100 or the treatment tool 1400 by driving a built-in motor based on an operation input to the operation devices 1300 and 1310. Alternatively, when the motor is present outside the drive control device 1200, the drive control device 1200 transmits a control signal to the external motor based on an operation input to the operation devices 1300 and 1310, thereby controlling the electrical driving. In addition, the drive control device 1200 may drive a built-in pump or the like based on an operation input to the operation device 1300, thereby causing the endoscope 1100 to perform air supply and/or suction. The air supply and/or suction are performed through an air supply/suction tube provided in the internal route 1101. One end of the air supply/suction tube opens to the distal end section 1130 of the endoscope 1100, while the other end is connected to the drive control device 1200 via the connector 1201. In addition, the treatment tool channel may be extended to the connector 1201, and the treatment tool channel may also be used as an air supply/suction tube.

Although the overtube 1710 and the balloon 1720 of FIG. 23 are omitted in FIG. 35, the overtube 1710 and the balloon 1720 may be used also in FIG. 35.

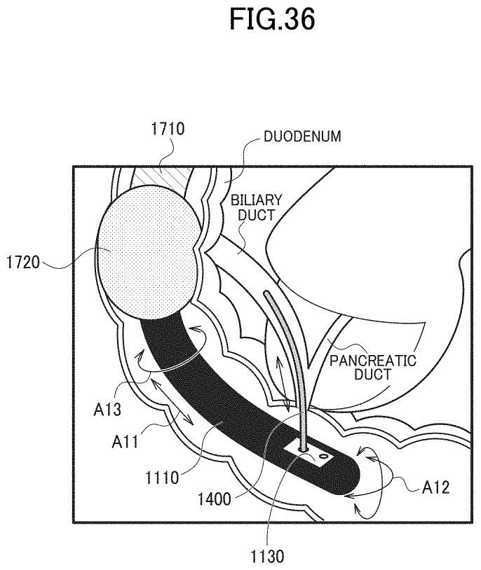

FIG. 36 shows the vicinity of the distal end of an endoscope positioned at a papillary portion of duodenum. FIG. 36 shows an example using the overtube 1710 and the balloon 1720.

As shown in FIG. 36, the balloon 1720 is fixed at a position slightly apart from the papillary portion to the pyloric side of the stomach. More specifically, the balloon 1720 is positioned closer to the base end of the insertion section 1110 than the base end of the bending section of the insertion section 1110. The endoscopic operation by the electrical driving is the forward and backward movement shown by arrow A11, a bending movement shown by arrow A12, or a rolling rotation shown by arrow A13. The forward movement is a shift toward the distal end side along the axial direction of the insertion section 1110, and the backward movement is a shift toward the base end side along the axial direction of the insertion section 1110. The bending movement is a movement by which the angle of the distal end section 1130 is changed due to the bending of the bending section. The bending movement includes bending movements in two orthogonal directions, which can be controlled independently. One of the two orthogonal directions is referred to as the vertical direction and the other is referred to as the horizontal direction. The rolling rotation is a rotation about an axis of the insertion section 1110. The meaning is the same also for the forward and backward movement, the bending, and the rolling rotation of the treatment tool.

FIGS. 37a-37c show a detailed configuration example of a forward/backward drive device. Although the forward/backward drive device 1800 for an endoscopes is described herein as an example, the forward/backward drive device 1460 for a treatment tool has a similar structure. The forward/backward drive device 1800 includes a motor 1816, a base 1818, and a slider 1819.

As shown in FIGS. 37a and 37b, the extracorporeal soft section 1140 of the endoscope 1100 is provided with an attachment 1802 detachable from the motor 1816. As shown in FIG. 37b, the attachment of the attachment 1802 to the motor 1816 enables electrical driving of forward/backward movement. As shown in FIG. 37c, the slider 1819 supports the motor 1816 while enabling the motor 1816 to move linearly with respect to the base 1818. The slider 1819 is fixed to the operating table T1 shown in FIG. 35. As shown by arrow B11, the drive control device 1200 transmits a forward or backward control signal to the motor 1816 by wireless communication, and the motor 1816 and the attachment 1802 move linearly on the slider 1819 based on the control signal. As a result, the forward and backward movement of the endoscope 1100 shown by arrow A11 in FIG. 40 is achieved. Note that the drive control device 1200 and the motor 1816 may be connected by wired connection.

FIGS. 38a and 38b show a detailed configuration example of a distal end section 1130 of an endoscope including a raising base of a treatment tool. FIG. 38a shows an external view of the distal end section 1130. An opening 1131 of a treatment tool channel, a camera 1132, and an illumination lens 1133 are provided on the side surface of the distal end section 1130. As shown in FIG. 38b, the direction parallel to the axial direction of the distal end section 1130 is defined as z1 direction, the direction parallel to the line-of-sight direction of the camera 1132 is defined as y1 direction, and the direction orthogonal to the z1 direction and the y1 direction is defined as x1 direction. FIG. 38b shows a cross-sectional view of the distal end section 1130 in a plane that is parallel to the y1z1 plane of the treatment tool channel and that passes through the opening 1131 of the treatment tool channel.

The distal end section 1130 includes a raising base 1134 and a raising base wire 1135. The raising base 1134 is swingable about an axis parallel to the x1 direction. One end of the raising base wire 1135 is connected to the raising base 1134, while the other end is connected to the drive control device 1200 via the connector 1201. As shown by arrow B14, the wire drive section 1250 of the drive control device 1200 pushes and pulls the raising base wire 1135 to swing the raising base 1134, thereby, as shown by arrow A14, changing the raising angle of the treatment tool 1400. The raising angle is an angle of the treatment tool 1400 protruding from the opening 1131. The raising angle can be defined, for example, by an angle formed by the treatment tool 1400 protruding from the opening 1131 and the z1 direction.

As explained above, in order to properly perform the procedure such as cannulation in ERCP, the sense of feel in pushing and pulling the sheath of the treatment tool by hand is important. However, when the treatment tool is electrically driven, the electric treatment tool is operated by operating the operation device instead of manually operating the sheath of the treatment tool by hand. Commonly used operation input sections include buttons, dials, D-pads, levers, and the like. However, these operation input sections are incapable of reproducing the sense of feel in pushing and pulling the sheath of the treatment tool by hand. Although the prior art may disclose a robotic catheter system, it does not disclose or suggest any of the above-mentioned problems or subject matter for solving them.

Therefore, the medical system 1010 of the present embodiment includes the treatment tool 1400, the operation device 1300, and the control device 1600. The treatment tool 1400 is inserted into the endoscope 1100 and includes a sheath whose forward and backward movement is electrically driven. The operation device 1310 is used to operate the electrically-driven forward and backward movement of the sheath. The operation device 1310 includes the base 1314, the operation sheath 1315 longitudinally slidable with respect to the base 1314, and the sliding operation detection sensor 1332 for detecting the sliding operation of the operation sheath 1315. The control device 1600 controls the electrically-driven forward and backward movement of the sheath of the treatment tool 1400 based on detected information of the sliding operation detected by the sliding operation detection sensor 1332.

According to the present embodiment, the operator can operate the forward and backward movement of the electric treatment tool by pushing and pulling the operation sheath 1315, which is a replica of the sheath of the treatment tool 1400, in the axial direction. As a result, the sense of feel in causing the forward and backward movement of the non-electric treatment tool can be reproduced in the operation device 1310 of the electric treatment tool, thereby obtaining a natural sense of feel in operation in the procedure, such as cannulation.

The electric treatment tool is described, for example, with regard to FIG. 24. In this example, the sheath is the tube 1421. The structure in which the forward and backward movement of the sheath is controlled based on the operation device, the base, the operation sheath, and the detected information of the sliding operation is described above.

Further, in the present embodiment, the operation device 1310 may include a force feedback section 1333 that performs force feedback with respect to the sliding operation. The control device 1600 may perform a force feedback control that causes the force feedback section to perform force feedback according to a reaction force that occurs when the distal end section of the treatment tool 1400 comes in contact with a tissue.

According to the present embodiment, the reaction force when the distal end section of the treatment tool 1400 comes in contact with a tissue is fed back to the operation sheath 1315, thereby reproducing the hand sensation in operating a non-electric treatment tool in the operation device 1310 of an electric treatment tool. Also, by performing the force feedback with respect to the operation sheath 1315, which is a replica of the sheath of the treatment tool 1400, a natural force feedback similar to when operating a non-electric treatment tool can be obtained.

The force feedback is described above. Insofar as the force feedback is made according to the reaction force when the distal end section of the treatment tool comes in contact with a tissue, detection of the reaction force itself is not necessary. That is, it is sufficient that the physical quantity of a change caused by the reception of the reaction force from the tissue at the distal end section of the treatment tool is detected, and the force feedback is controlled based on the detection result.

Further, in the present embodiment, the treatment tool 1400 may also include the force senser 1480, which is disposed in the distal end section of the sheath. The control device 1600 may perform the force feedback control based on detected force information detected by the force sensor 1480.

According to the present embodiment, the reaction force when the distal end section of the treatment tool comes in contact with a tissue is detected by the force senser 1480, and the force feedback control is performed based on the detected force information. That is, the reaction force received from the tissue at the distal end section of the treatment tool is directly detected as a sense of force.

The force feedback using the force senser is described with regard to FIG. 26 and FIG. 28.

Further, in the present embodiment, the control device 1600 may also perform the force feedback control based on an endoscope image, which is an image showing the sheath captured by the endoscope 1100.

According to the present embodiment, reception of a reaction force from a tissue at the distal end section of the treatment tool changes the movement of the sheath shown in the endoscope image, and the force feedback control is performed based on the change. That is, the reaction force received from a tissue at the distal end section of the treatment tool is indirectly detected from the endoscope image.

The force feedback using an endoscope image is described with regard to FIG. 30.

Further, in the present embodiment, the base 1314 may be disposed on one end of the operation sheath 1315. The operation device 1310 may include the sheath holding section 1317, which holds the operation sheath 1315 at the other end of the operation sheath 1315.

According to the present embodiment, the base 1314 and the sheath holding section 1317 hold both ends of the operation sheath 1315, allowing the operator to perform the sliding operation of the operation sheath 1315 provided between these ends. Further, holding both ends of the operation sheath 1315 stabilizes the operation sheath 1315, allowing the operator to perform the operation more easily.

The sheath holding section is described with regard to FIG. 25.

Further, in the present embodiment, the control device 1600 may perform the force feedback control such that the sliding operation reaction force against the sliding operation increases as the reaction force increases.

In the case of operating a non-electric treatment tool by hand, the reaction force received from a tissue at the distal end section of the treatment tool is transmitted to the hand via the sheath. That is, the larger the reaction force, the greater the sense of force felt in the hand. According to the present embodiment, the larger the reaction force, the larger the sliding operation reaction force. This enables reproduction of the sense of feel in operating a non-electric treatment tool by hand.

The relationship between the reaction force received from a tissue at the distal end section of the treatment tool and the sliding operation reaction force is described with regard to FIG. 29 and FIG. 30.

Further, in the present embodiment, the control device 1600 may also change the determination as to whether or not the force feedback control is performed, according to the type of the treatment tool 1400.

According to the present embodiment, the force feedback can be enabled or disabled according to the type of the treatment tool. For example, it may be arranged such that the force feedback is enabled in a treatment tool with a force feedback function. It may also be arranged such that the force feedback is enabled or disabled depending on the type of the treatment tool, such as cannula, electronic knife, guide wire, basket, or stent, or depending on the lineup, variation, grades, etc. of the treatment tool.

The change of determination as to whether the force feedback control is performed according to the type of the treatment tool is described with regard to FIG. 32.

Further, in the present embodiment, the treatment tool 1400 may be a cannula. Further, in the present embodiment, the treatment tool 1400 may be a treatment tool to be inserted into a biliary duct or a pancreatic duct. The control device 1600 may electrically drive the sliding operation when the treatment tool 1400 is inserted into a biliary duct or a pancreatic duct after the distal end section 1130 of the endoscope 1100 is positioned at a papillary portion of duodenum.

During the cannulation in ERCP, by operating the forward and backward movement of an electric cannula using the operation device 1310 of the present embodiment, it is possible to reproduce the sense of feel in performing the forward and backward movement of a non-electric cannula. In the cannulation, since the cannula is inserted into the biliary duct, which is not shown in the endoscope image, the sense of feel in operation is important. According to the present embodiment, the sense of feel can be reproduced in an electric cannula.

The flow of ERCP procedure using an electric treatment tool is described with regard to FIG. 31.

Further, in the present embodiment, the operation sheath 1315 may be tiltable in a direction different from the sliding direction. The control device 1600 may change the direction of the raising base 1134 by which the treatment tool 1400 is raised when the operation sheath 1315 is tilted in a direction different from the sliding direction.

According to the present embodiment, the operation device 1310 for the treatment tool enables operation of the raising angle, together with the forward and backward movement of the treatment tool. In the cannulation, in some cases, the sheath is moved forward and backward while adjusting the raising angle; therefore, it is desirable to perform the movement and the adjustment by a single device, i.e., the operation device 1310.

The operation device for the treatment tool capable of operating the raising angle is described with regard to FIG. 33. The operation sheath being the tiltable means that the operation sheath is tiltable with respect to the base or a part of the base. In the example in FIG. 33, since the first base 1311 is tiltable with respect to the second base 1312, the operation sheath 1315 held by the first base 1311 is also tiltable with respect to the second base 1312.

Further, in the present embodiment, the medical system 1010 may include an endoscope operation device 1300 for operating the electrically-driven endoscopic operation of the endoscope 1100. The operation device 1310 may include an attachment 1319 for allowing the operation device 1310 for operating the treatment tool 1400 to be detachably attached to the endoscope operation device 1300.

According to the present embodiment, the attachment 1319 allows the operation device 1310 for the treatment tool to be integrated with the operation device 1300 for the endoscope, thereby operating the endoscope and the treatment tool by the integrated operation device. Further, with the detachable structure, it becomes possible to change the operation device according to the type of the treatment tool, and attach it to the operation device 1300 for the endoscope for use.

Further, in the medical system 1010, the electrical driving of the bending movement of the endoscope 1100 is not limited to the structure of the present embodiment. For example, it may be structured such that an attachment equipped with an electric motor is detachably attached to a bending operation knob of a non-electrically-driven endoscope. The drive control device 1200 and the attachment are structured to communicate with each other, and, upon reception of a bending control signal from the drive control device 1200, the attachment is driven to perform the bending. In this case, the manual control and the automatic control can be switched by attaching and detaching the attachment. It may also be arranged such that a handle capable of controlling the driving of the drive control device 1200 is detachably attached to a motor for bending control corresponding to the drive control device 1200. In this case, the manual control and the automatic control can be switched by attaching and detaching the handle.

According to an aspect of the disclosed embodiments, the following can be provided.

1. A medical system comprising:

a treatment tool configured to be inserted into an endoscope and including a sheath whose forward and backward movement is electrically driven;

an operation device for operating the electrically-driven forward and backward movement of the sheath; and

a controller comprising hardware,

the operation device comprising:
a base;
an operation sheath disposed to be longitudinally slidable with respect to the base; and
a sliding operation detection sensor configured to detect a sliding operation of the operation sheath,

the controller being configured to control the electrically-driven forward and backward movement of the sheath of the treatment tool based on detected information of the sliding operation detected by the sliding operation detection sensor.

2. The medical system as defined in claim 1, wherein

the operation device includes a force feedback section configured to perform force feedback in response to the sliding operation, and

the controller performs a force feedback control that causes the force feedback section to perform the force feedback according to a reaction force that occurs when a distal end section of the treatment tool contacts tissue.

3. The medical system as defined in claim 2, wherein

the treatment tool includes a force senser disposed at a distal end section of the sheath, and

the controller performs the force feedback control based on detected force information detected by the force sensor.

4. The medical system as defined in claim 2, wherein

the controller performs the force feedback control based on an endoscope image showing the sheath captured by the endoscope.

5. The medical system as defined in claim 2, wherein

the controller performs the force feedback control such that a sliding operation reaction force against the sliding operation increases as the reaction force increases.

6. The medical system as defined in claim 2, wherein

the controller changes determination as to whether or not to perform the force feedback control according to a type of the treatment tool.

7. The medical system as defined in claim 1, wherein

the base is disposed on one end of the operation sheath; and

the operation device includes a sheath holding section for holding the operation sheath on an other end of the operation sheath.

8. The medical system as defined in claim 1, wherein

the treatment tool is a cannula.

9. The medical system as defined in claim 1, wherein

the treatment tool is configured to be inserted into a biliary duct or a pancreatic duct, and

the controller electrically drives the sliding operation when the treatment tool is inserted into the biliary duct or the pancreatic duct after positioning of a distal end section of the endoscope at a papillary portion of duodenum.

10. The medical system as defined in claim 1, wherein

the operation sheath is tiltable in a direction different from the sliding direction, and the controller changes a direction of a raising base by which the treatment tool is raised when the operation sheath is tilted in the direction different from the sliding direction.

11. The medical system as defined in claim 1, further comprising an endoscope operation device for operating an electrically-driven endoscopic operation of the endoscope,

wherein

the operation device includes an attachment that allows the operation device for operating the treatment tool to be detachable from the endoscope operation device.

Although the embodiments applied and the modifications thereof have been described above, the invention is not limited to the embodiments and the modifications thereof, and various modifications and variations in elements may be made in implementation without departing from the spirit and scope of the present disclosure. The plurality of elements disclosed in the embodiments and the modifications described above may be combined as appropriate to form various disclosures. For example, some of all the elements described in the embodiments and the modifications may be deleted. Furthermore, elements in different embodiments and modifications may be combined as appropriate. Thus, various modifications and applications can be made without departing from the spirit and scope of the invention. Any term cited with a different term having a broader meaning or the same meaning at least once in the specification and the drawings can be replaced by the different term in any place in the specification and the drawings.

What is claimed is:

1. A medical system comprising:
an endoscope configured to electrically drive an endoscopic operation, the endoscopic operation comprising at least one of a forward and backward movement of an insertion section, a bending angle of a bending section of the insertion section, and a rolling rotation of the insertion section, the endoscope being configured to capture an endoscope image; and
a processor comprising hardware, the processor being configured to control the endoscopic operation to achieve a second positioning subsequent to a first positioning, the first positioning being where the insertion section is positioned with respect to a papillary portion of a duodenum, the second positioning positions a distal end section of the insertion section with respect to a papillary portion of the duodenum based on the endoscope image.

2. The medical system of claim 1, further comprising a holding member configured to hold the insertion section relative to the duodenum to achieve the first positioning of the insertion section.

3. The medical system of claim 2, wherein the second positioning comprises adjusting a position of the distal end section of the insertion section on a distal end side of the insertion section relative to the holding member holding the insertion section.

4. The medical system of claim 1, wherein the second positioning comprises controlling the endoscopic operation such that an image of the papillary portion is captured at a position registered in advance on the endoscope image.

5. The medical system of claim 1, where the papillary portion includes an opening of a luminal tissue, which is an opening of a common duct in which a biliary duct and a pancreatic duct merge or an opening of a biliary duct, the medical system further comprising:
a treatment tool configured to be inserted from an insertion opening of a treatment tool channel of the endoscope, the treatment tool being raised inside the distal end section to protrude from a side surface of the distal end section, wherein
the second positioning comprising controlling the endoscopic operation and a raising angle of the treatment tool such that the treatment tool faces toward a travelling direction of the biliary duct that is presumed from the endoscope image in which the image of the papillary portion is captured.

6. The medical system of claim 2, wherein the holding member is provided closer to a base end of the insertion section than a base end of the bending section.

7. The medical system of claim 2, wherein the holding member is a balloon that is inflated and comes in contact with the duodenum so as to hold the insertion section with respect to the duodenum.

8. The medical system of claim 2, wherein the holding member is a first holding member, the medical system further comprises a second holding member for holding a route of the insertion section.

9. The medical system of claim 8, further comprising an overtube having the second holding member, the overtube being configured to have a variable hardness that holds the route of the insertion section by being hardened.

10. A cannulation method using an endoscope configured to electrically drive an endoscopic operation, is the endoscopic operation comprising at least one of a forward and backward movement of an insertion section, a bending angle of a bending section of the insertion section, and a rolling rotation of the insertion section, the endoscope being configured to capture an endoscope image, the cannulation method comprising,
inserting the insertion section of the endoscope into a body;
performing a first positioning of the insertion section with respect to a papillary portion of a duodenum;
subsequent to the first positioning, performing a second positioning of a distal end section of the insertion section with respect to the papillary portion by controlling the electrically-driven endoscopic operation based on the endoscope image; and
subsequent to the second positioning, performing cannulation from the papillary portion to a biliary duct.

11. The cannulation method of claim 10, wherein the first positioning comprises holding the insertion section relative to the duodenum.

12. The cannulation method of claim 11, wherein the second positioning comprises adjusting a position of the distal end section of the endoscope by the endoscopic operation on a distal end side relative to the holding member holding the insertion section.

13. The cannulation method of claim 10, wherein the second positioning comprises controlling the electrically-driven endoscopic operation such that an image of the papillary portion is captured at a position registered in advance on the endoscope image.

14. The cannulation method of claim 10, wherein the second positioning comprises controlling the endoscopic operation and a raising angle of the treatment tool such that a treatment tool that is inserted from an insertion opening of a treatment tool channel of the endoscope and is raised inside the distal end section to protrude from a side surface of the distal end section to face toward a travelling direction of a biliary duct that is presumed from the endoscope image in which an image of the papillary portion is captured.
